(19) United States
(12) Patent Application Publication
(10) Pub. No.: US 2017/0035860 A1
(43) Pub. Date: Feb. 9, 2017

(54) COMPOSITIONS AND METHODS FOR TREATMENT OF NEUROGENERATIVE DISEASES

(71) Applicant: Alexander C. Flynn, Houston, TX (US)

(72) Inventor: Alexander C. Flynn, Houston, TX (US)

(21) Appl. No.: 15/089,174

(22) Filed: Apr. 1, 2016

Related U.S. Application Data
(60) Provisional application No. 62/142,243, filed on Apr. 2, 2015.

(51) Int. Cl. A61K 38/46; C12N 9/22; A61K 47/26

(52) U.S. Cl. CPC A61K 38/465; A61K 47/26; C12N 9/22; A61K 48/00

(57) ABSTRACT

Medical compositions and methods of treating or preventing neurodegeneration in a human suffering from or that is at risk of or susceptible to neurodegeneration or cellular dysfunction associated with expression or impaired cellular function of a neuronal protein encoded by one or more genes that code for alpha-synuclein (SNCA), Parkin RBR E3 ubiquitin protein ligase, (PARK2), Leucine-rich repeat kinase 2 (LRRK2), PTEN-induced putative kinase/(PINK1), Daisuke-Junko 1, (DJ-1) and ATPase type 13A2 (ATP13A2), are disclosed. Methods of treatment for these disorders is also provided, comprising administering a vector into a cell, wherein the vector facilitates expression of a molecular component that alters one of the aforementioned genes in the cell or expression of the gene in the cell, the gene being implicated in an etiology of the neurological deficit.

### CROSS-REFERENCE TO RELATED APPLICATIONS

The present application claims priority to U.S. Provisional Application Ser. No. 62/142,243, filed Apr. 2, 2015. The entire contents of U.S. Provisional Application Ser. No. 62/142,243 is incorporated herein in its entirety.

### FIELD OF THE INVENTION

The present invention generally relates to the field of therapeutics and therapeutic methods using genetic manipulation, as therapeutics and therapeutic methods that provide for genetically modifying one or several gene sequences in a population of human cells to be used as a therapeutic and/or therapeutic method for the treatment of a neurological disorder (e.g., Parkinson's disease) are disclosed. The invention also relates to the field of protein expression modulation, as methods for providing altered protein expression through selected techniques and modifications at the DNA level are demonstrated that provide for the peunanent arrest of disease progression.

The invention also generally relates to the compositions and methods of genetically modifying one or several gene sequences in human cells in vivo; either in coding sequences of a gene (creating random mutations at defined positions or insert exogenous genomic sequence to introduce defined changes in the gene), or alteration of regulatory elements that regulate gene expression for selected genes causative for or which increase the risk for neurodegeneration. The gene modification methods provided in the present invention may permanently alter or transiently affect the genomic sequence of a gene. Hence, a method to provide modified gene sequences and altered protein expression is provided, wherein progression of disease is permanently arrested.

### BACKGROUND OF THE INVENTION

Parkinson's disease (PD) and Alzheimer's disease (AD) are the two most common neurodegenerative diseases of aging. Approximately 1-2% of the population over 65 years of age are affected by PD, and it is estimated that the number of prevalent cases of this disease will double by the year 2030 (Dorsey et al., 2007). PD has also been observed in younger patients, possibly due to specific genetic susceptibility and/or exposure to environmental factors, drugs, or trauma. PD is a slowly progressive debilitating disease with no known current cure. The cause (or causes) of PD also remain largely unknown. While the classical clinical features of the disease include tremor, rigidity, bradykinesia, and postural instability (Gelb et al., 1999) it is now increasingly clear that the disease comprises a whole spectrum of clinical symptoms that go far beyond motor deficits, including reduction in sense of smell, sleep problems, autonomic dysfunction, psychiatric abnormalities, and cognitive decline, which are all part of the clinical syndrome (Langston, 2006). Furthermore, the disease exhibits a remarkable degree of clinical variability in terms of severity, age at onset, and gender difference, with a male/female ratio of approximately 2:1 (Van Den Eeden et al., 2003).

Although PD represents mostly a sporadic disease, familial forms of parkinsonism and several causative genes have been described and investigated (reviewed in Klein and Schlossmacher, 2007; Klein and Westenberger, 2012; Puschmann, 2013 Table 1 and 2). Neuropathologically, the cardinal features of PD include loss of dopaminergic neurons in the substantia nigra of the brain, and the buildup of intracellular inclusions known as Lewy bodies, the neuropathological hallmark of PD. The presence of Lewy bodies is classically associated with dementia (DLB)(Poulopoulos et al., 2012; Schulz-Schaeffer, 2010). A major component of Lewy bodies is the protein, alpha-synuclein. Alpha-synuclein (approved gene symbol is SNCA) was the first gene in which a causative mutation, SNCA p. A53T, was discovered in 1997 (Polymeropoulos et al., 1997). With the advent of alpha-synuclein based immunohistochemistry, a staining method to detect this protein within a cell, it has been possible to better characterize not only Lewy bodies, but also the wide-spread alpha-synuclein positive Lewy neuritic pathology that exists in both the central and peripheral nervous system. These tools have provided an increasingly well-defined neuroanatomical basis for the many non-motor features of this disease (Forman et al., 2005; Langston, 2006; Litvan et al., 2007a; Litvan et al., 2007b).

Other alpha-synucleinopathies have been described that present a spectrum of disorders having a range of clinical symptoms, such as Parkinson's disease with dementia (PDD), dementia with Lewy bodies (DLB), multiple system atrophy (MSA), atypical parkinsonism, and atypical Parkinson's disease (Table 2).

The use of small interference RNAs (siRNA) in the brain has recently been shown to be effective against endogenous murine alpha-synuclein (Gorbatyuk et al., 2010; Khodr et al., 2014; Lewis et al., 2008) serotonin transporter (SERT) (Ferres-Coy et al., 2013), and mutant human Huntingtin (Chen et al., 2005; DiFiglia et al., 2007; Pfister et al., 2009). siRNA knockdown of alpha-synuclein has also been shown in an MPTP-exposed non-human primate (McCormack et al., 2010). Alpha-synuclein siRNA knockdown in animals has been reported to provide neuroprotection in non-human primates against MPTP. In this model, downregulation of alpha-synuclein mRNA and protein was observed to protect neurons from degeneration and cell death (Maraganore, 2011).

The advent of alpha-synuclein based immunohistochemistry, a staining method to detect protein within a cell, resulted in the report that the protein, alpha-synuclein, is directly linked to the pathogenesis of certain forms of familial parkinsonism, as well as sporadic PD.

The protein name is alpha-synuclein, whereas the corresponding approved gene name abbreviation is SNCA. Genetic changes of the SNCA gene, which are point mutations and large genomic multiplications, can cause familial PD (Chartier-Harlin et al., 2004; Fuchs et al., 2007a; Ibanez et al., 2004; Krüger et aL, 1998; Nishioka et al., 2006; Polymeropoulos et al., 1997; Singleton et al., 2003; Zarranz et al., 2004). Two point mutations have been described to be causative for PD, SNCA p.H50G, and SNCA, p.G51D. This raises the number of SNCA point mutations associated with PD to five (Appel-Cresswell et al., 2013; Kiely et al., 2013; Lesage et al., 2013; Proukakis et al., 2013). The dominant inheritance of SNCA mutations, or the mere overexpression of wild-type alpha-synuclein, suggest that parkinsonism is caused by a toxic gain-of-function mechanism. This means that in the disease, the gene/protein takes on a different function that is harmful to the cell. Also, the finding that both qualitative and quantitative alterations in the SNCA gene are associated with the development of a parkinsonian phenotype indicates that amino acid substitutions and overexpression of wild-type alpha-synuclein are capable of triggering a clinicopathological process that is very similar to typical PD (Deng and Yuan, 2014), including nigrostriatal cell death and intracellular protein aggregations, called Lewy bodies.

One hypothesis regarding the cause of PD is that subtle to moderate overexpression of alpha-synuclein due to other genetic risk variants over many decades can either predispose or even cause the neurodegenerative changes that characterize PD similar to SNCA gene multiplications linked to PD. Several association studies investigated specific polymorphisms within the SNCA gene that might be expected to alter its expression, and have been reported to be associated with PD (Chiba-Falek et al., 2005; Chiba-Falek and Nussbaum, 2001; Chiba-Falek et al., 2003; Farrer et al., 2001; Holzmann et al., 2003; Maraganore et al., 2006; Mizuta et al., 2006; Pals et al., 2004; Wang et al., 2006). The NACP-Rep1 polymorphism of the SNCA promoter, a mixed dinucleotide repeat, was studied in multiple study populations, and reports an association of the NACP-Rep1 allele with PD (Farrer et al., 2001; Hadjigeorgiou et al., 2006; Krüger et al., 1999; Mellick et al., 2005; Pals et al., 2004; Tan et al., 2003). It was also reported that the NACP-Rep1 alleles differed in frequency for cases and controls (P<0.001) and the long allele, 263 bp, was associated with PD (odds ratio, 1.43) (Maraganore et al. et al., 2006).

The DNA binding protein and transcriptional regulator, PARP-1, showed specific binding to SNCA-Rep1. The PARPs catalyze the transfer of ADP-ribose to various nuclear proteins, and are involved in several cellular processes (e.g., DNA repair, regulation of chromatin structure, transcriptional regulation, trafficking, cell death activation (Beneke and Burkle, 2007)). These data were confirmed by a transgenic mouse model and demonstrated regulatory translational activity (Cronin et al., 2009). Functionally, SNCA expression levels in postmortem brains suggest that the Rep-1 allele and SNPs in the 3′ region of the SNCA gene have a significant effect on SNCA mRNA levels in the substantia nigra and the temporal cortex.

The promoter region of the SNCA gene has been examined in cancer cell lines and in rat cortical neurons. Regulatory regions in intron 1 and the 5′ region of exon 1 have been shown to exhibit transcriptional activation (Clough et al., 2011; Clough et al., 2009; Clough and Stefanis, 2007), as well as the NACP-Rep-1 region upstream of the SNCA gene (Chiba-Falek et al., 2005; Chiba-Falek and Nussbaum, 2001; Chiba-Falek et al., 2003; Cronin et al., 2009; Touchman et al., 2001). Several transcription factors have been identified, such as PARP-1 (Chiba-Falek et al., 2005), GATA (Scherzer et al., 2008), ZIPRO1, and ZNF219 (Clough et al., 2009) to have an effect on regulating the SNCA promoter region.

Gene isoforms of alpha-synuclein: Alpha-synuclein has three smaller gene isoforms, which have been described as a result of alternative mRNA splicing. Alpha-synuclein 140 is the main complete protein, alpha-synuclein 112 and alpha-synuclein 126 are shorter proteins lacking exon 5 (C-terminus) and exon 3 (N-terminus), respectively (Campion et al., 1995; Ueda et al., 1993). A third isoform is lacking both exon 3 and exon 5, resulting in a protein product of 96 amino acids (Beyer et al., 2008). It has been shown that each of the three alternative isoforms aggregates significantly less than the canonical isoform SNCA140 (Bungeroth, M., Appenzeller, S., Regulin, A., Volker, W., Lorenzen, I., Grotzinger, J., Pendziwiat, M., and Kuhlenbaumer, G. (2014). Differential aggregation properties of alpha-synuclein isoforms. Neurobiol Aging 35, 1913-1919.)).

Beyer and colleagues examined expression levels of the alpha-synuclein isoforms 112, 126, and 140 in patients with Diffuse Lewy body disease (DLB), Alzheimer disease (AD), and controls by quantitative RT-PCR. Overall, total alpha-synuclein was upregulated in patients with DLB compared to controls. The relative expression of alpha-synuclein 112 in patients with DLB showed a significant increase compared to controls, whereas isoform 126 had surprisingly lower levels in synucleinopathies and AD compared to controls (Beyer, 2006; Beyer et al., 2006; Beyer et al., 2004). The shift expression ratios for the alpha-synuclein isoforms could be crucial for the disease process and neurodegeneration as shown for the tau gene (Hutton et al., 1998). The importance of isoform ratios shows the misregulation of the 4R to 3R isoforms of tau in familial fronto-temporal dementia with parkinsonism (FTDP-17). A balance between these isoforms seems to be highly important for neuronal function. Mutations of exon 10 of the tau gene either include or exclude exon 10 and alter the 3R14R ratio leading to the disease phenotype (Hong et al., 1998; Hutton et al., 1998). Specht and collaborators evaluated alpha-synuclein—eGFP fusion proteins in vitro to identify functional protein motifs. Cytoplasmic distribution was observed in two N-terminal constructs, which showed some similarity to the alpha-synuclein isoform 112. Constructs of the C-terminal domain of alpha-synuclein caused almost exclusively nuclear localization (Specht et al., 2005).

Kontopoulos and coworkers report that a vector construct expressing alpha-synuclein tagged with a nuclear localization signal may mediate neurotoxicity, whereas its cytoplasmic localization might be protective in an in vitro system and transgenic Drosophila (Kontopoulos et al., 2006).

Specific subcellular distribution of alpha-synuclein can exhibit differences in toxicity. Predominately cytoplasmic distribution is neuroprotective whereas nuclear distribution is neurotoxic. Shifting the distribution of alpha-synuclein towards the cytoplasm by creating a specific alpha-synuclein isoform with 112 amino acids is a neuroprotective or neuroregenerative approach.

Gene editing technologies: Recent developments of technologies to permanently alter the human genome and to introduce site-specific genome modifications in disease relevant genes lay the foundation for therapeutic applications in CNS disorders such as Parkinson's disease (PD) or Alzheimer disease (AD). These technologies are now commonly known as “genome editing.” Current gene editing technologies comprise zinc-finger nucleases (ZFN), TAL effector nucleases (TALEN), and clustered regularly interspaced short palindromic repeats (CRISPR)/CRISPR-associated (Cas) system or a combination of nucleases (e.g. mutated Cas9 with Fok1) (Tsai, S. Q., Wyvekens, N., Khayter, C., Foden, J. A., Thapar, V., Reyon, D., Goodwin, M. J., Aryee, M. J., and Joung, J. K. (2014). Dimeric CRISPR RNA-guided Fold nucleases for highly specific genome editing. Nature biotechnology 32, 569-576.)) All three technologies create site-specific double-strand breaks. The imprecise repair of a double strand break by non-homologous end joining (NHEJ) has been used to attempt targeted gene alteration (nucleotide insertion, nucleotide deletion, and/or nucleotide substitution mutation). A double-strand break increases the frequency of homologous recombination (HR) at the targeted locus by 1,000 fold, an event that introduces homologous sequence at a target site, such as from a donor DNA fragment. Another approach to minimize off-target effects is to only introduce single strand breaks or nicks using Cas9 nickase (Chen et al., 2014; Fauser et al., 2014; Rong et al., 2014; Shen et al., 2014).

The CRISPR/Cas9 nuclease system can be targeted to specific genomic sites by complexing with a synthetic guide RNA (sgRNA) that hybridizes a 20-nucleotide DNA sequence (protospacer) immediately preceding an NGG motif (PAM, or protospacer-adjacent motif) recognized by Cas9. CRISPR-Cas9 nuclease generates double-strand breaks at defined genomic locations that are usually repaired by non-homologous end-joining (NHEJ). This process is error-prone and results in frameshift mutation that leads to knock-out alleles of genes and dysfunctional proteins (Gilbert et al., 2013; Heintze et al., 2013; Jinek et al., 2012). Studies on off-target effects of CRISPR show high specificity of editing by next-generation sequencing approaches (Smith et al., 2014; Veres et al., 2014) (FIG. 1, panel 1).

Other applications for heart disease, HIV, and Rett syndrome have been described. (Ding et al., 2014; Swiech et al., 2014; Tebas et al., 2014). For heart disease, permanent alteration of a gene called PCSK9 using CRIPR technology reduces blood cholesterol levels in mice (Ding et al., 2014). This approach was based on the observation that individuals with naturally occurring loss-of-function PCSK9 mutations experience reduced blood low-density lipoprotein cholesterol (LDL-C) levels and protection against cardiovascular disease (Ding et al., 2014). A second example for the feasibility of this approach is HIV. Individuals carrying the inherited Delta 32 mutation in the C-C chemokine receptor type 5, also known as CCR5 or CD195 are resistant to HIV-1 infection. Gene modification in CD4 T cells were tested in a safety trial of 12 patients and has shown a significant downregulation of CCR5 in human (Tebas et al., 2014). Another recent study showed the successful use of CRISPR/Cas9 technology in CNS in a mouse model for the editing of the methyl-binding protein 2 (MecP2) gene. Mutation in this gene causes Rett syndrome, a condition in young children—mostly girls—with mental retardation and failure to thrive. In this approach an adeno-associated virus (AAV) was used as the delivery vehicle for the Cas9 enzyme in vivo. Overall, 75% transfection efficiency was described with a high targeting efficiency that almost completely abolished the expression of MecP2 protein and functionally altered that arborization of the neurons similar to what has been described for Rett syndrome (Swiech et al., 2014). This shows the proof of concept that gene editing using CRISPR/Cas9 technology is achievable in the adult brain in vivo.

Despite reports in the literature describing the use of genetic editing techniques, none have been described or suggested for genes associated with neurodegenerative disorders. A strong need continues to exist in the medical arts for a method for treating and/or inhibiting diseases associated with neurodegenerative disorders, such as materials and techniques useful for the treatment of Parkinson's Disease.

### SUMMARY OF THE INVENTION

In a general and overall sense, the present invention provides for the arrest and/or prevention of neurodegeneration associated with neurodegernative disease in vivo. In some embodiments, arrest and/or prevention of neurodegeneration is accomplished using gene editing methodologies and molecular tools to manipulate specific gene(s) and/or gene regulatory elements, to provide a modification of the gene and/or genomic regions associated with neurodegeneration and neurodegenerative disease, such as Parkinson's Disease.

In some aspects, the present invention provides a method of treating a neurological deficit associated with neuropathological disease comprising administering a genetically engineered vector comprising a gene for a nuclease and a promoter for the nuclease, as well as an appropriate molecular “guide” into a cell. Following the administration, the vector facilitates an expression of a molecular component that alters a gene in the cell or expression of a targeted gene associated with the neuropathology in the cell. The affected gene would be implicated in an etiology of the neurological deficit.

In other embodiments, a medical composition for treating a neurological deficit in a patient is provided. The medical composition includes a nuclease that introduces double strand break in a gene implicated a neurological deficit, a guide RNA that targets a gene implicated in neurological disease, and a delivery system that delivers the nuclease and guide RNA to a cell.

For purposes of the description of the present invention, the term “modification of gene and/or genomic region” may be interpreted to include one or more of the following events (FIG. 1):

a) Targeted introduction of a double-strand break by a composition disclosed, resulting in targeted alterations (random mutations e.g. insertions, deletions and/or substitution mutations) in one or more exons of one or more genes. This modification in some embodiments provides a permanent mutation in a cell or population of cells having the modified gene.

b) Targeted binding of non-functional mutant Cas9 to non-coding regions (e.g. promoters, evolutionary conserved functional regions, enhancer or repressor elements). Binding is induced by compositions disclosed. Sterical hindrance of binding of other proteins (e.g. transcription factors, polymerases or other proteins involved in transcription) may also result as a consequence of binding.

1. CRISPR sgRNA introduces small insertions or deletions through non-homologous end joining (NHEJ), in general several nucleotides, rarely larger fragments (Swiech et al., 2014).

2. Homology-directed repair (HDR) to correct point mutations by introducing a non-natural, but partially homologous template.

3. Double Genome editing of splice-sites or splicing related non-coding elements to eliminate certain gene regions, e.g. exon 5 of SNCA gene.

4. Double Genome editing of non-coding or intronic gene regions to eliminate regulatory elements that increase or decrease gene expression, e.g. D6 or I12 regulatory region in SNCA gene.

5. sgRNA guides mutant Cas9 to physically inhibit binding of transcription factors in promoter region,

6. sgRNA guides mutant Cas9 to physically inhibit binding of transcription factors in regulatory regions or intronically.

Gene editing or modification can be achieved by use of any variety of techniques, including zinc-finger nuclease (ZFN) or TAL effector nuclease (TALEN) technologies or by use of clustered, regularly interspaced, short palindromic repeat (CRIPSR)/Cas9 technologies or through the use of a catalytically inactive programmable RNA-dependent DNA binding protein (dCas9) fused to VP16 tetramer activation domain, or a Krueppel-associated box (KRAB) repressor domain, or any variety of related nucleases employed for gene editing. These can be seen as existing tools to sever the genomic region in question.

The tools mentioned above, are general in their application. Aspects of the present methods and compositions provide the design of custom CRISPR single-guide RNA (sgRNA) sequences specific for coding gene regions and regulatory sequences in genes implicated in neurodegeneration. In this manner, an exact genomic location for precise gene alteration in humans may be accomplished, with a resulting improvement and/or elimination of a neurodegenerative disorder pathology or symptom.

Other aspects of the invention provide for the use and accomplishment of the following molecular events, with the expected phenotypic results for neurodegenerative disease treatment:

1. CRISPR sgRNA leading to non-homologous end joining (NHEJ) introducing insertion or deletions (e.g. small nucleotide insertions or deletions resulting in frameshifts and non-functional protein) (FIG. 1, panel A).

2. CRISPR sgRNA with donor to achieve homology directed repair (HDR) to introduce a specific mutation or sequence change (FIG. 1, panel B).

3. Mutant Cas9/CRISPR sgRNA to target regulatory elements activating or repressing SNCA and/or MAPT gene expression, including promoter, enhancer elements, silencer elements or other like regulatory regions in cis or trans, or introns (FIG. 1, panel C).

4. CRISPR sgRNA to target splice sites to induce nonsense mediated decay or alteration of isoforms (FIG. 1, panel D).

5. sgRNA guides mutant Cas9 to physically inhibit binding of transcription factors in promoter region (FIG. 1, panel E).

6. sgRNA guides mutant Cas9 to physically inhibit binding of transcription factors in regulatory regions or intronically (FIG. 1, panel F).

Specific compositions of nucleotide sequences that can serve or be transcribed in vivo or in vitro into sgRNAs and can introduce changes.

In another aspect, specifically designed sgRNAs are provided that enhance promoter activity or other regulatory elements within a gene to enhance or repress expression of SNCA and/or MAPT or other genetic factors related to neurodegenerative disorders in vivo.

The sgRNA constructs may be applied alone or in combination to multiple specific sequences.

In some embodiments, specifically designed sgRNA sequences are provided that target or disrupt specific splice sites to preferentially create specific protein forms (isoforms) which prevent the promotion of protein aggregation, such as specifically targeted to the SNCA gene and/or MAPT gene.

In other embodiments, specifically designed donor constructs with specific, defined mutations to introduce frameshift mutations of genes implicated in neurodegenerative disease, leading to loss-of-function introduced by homology-directed repair (HDR), as provided.

The invention also provides specific promoter-driven expression of gene editing or modification constructs in defined cell populations, e.g. tyrosine hydroxylase promoter to deliver construct in defined cell populations, for selective delivery in targeted cell types that exhibit disease pathology.

In yet other embodiments, specific inducible/silencing constructs to turn on/off the expression of a gene modification construct are provided.

The invention includes various delivery systems for a vector, such as an adeno-associated virus (AAV) delivery system for in vivo/in human expression of gene editing modification machinery.

Editing of SNCA and tau patient-derived stem cells or human embryonic stem cells followed by transplantation of disease gene modified differentiated neurons (progenitors) from stem cells into human/patient is included in the present invention as part of a treatment method.

In some embodiments, gene editing or modification constructs described will be cloned in a single vector or multiple vectors.

Techniques and materials for gene editing or modification constructs delivered separately based on size of the constructs are also provided.

### BRIEF DESCRIPTION OF THE DRAWINGS

FIG. 1. Scheme of CRISPR Single-guide RNA design against gene coding and regulatory regions. 1A. CRISPR. sgRNA introduces small insertions or deletions through non-homologous end joining (NHEJ), in general several nucleotides, rarely larger fragments (Swiech et al., 2014), 1B. Homology-directed repair (HDR) to correct point mutations by introducing a non-natural, but partially homologous template (green), 1C. Double Genome editing of splice-sites or splicing related non-coding elements to eliminate certain gene regions, e.g. exon 5 of SNCA gene. 1D. Double Genome editing of non-coding or intronic gene regions to eliminate regulatory elements that increase or decrease gene expression, e.g. D6 or 112 regulatory region in SNCA gene. 1E. sgRNA guides mutant Cas9 to physically inhibit binding of transcription factors in promoter region, 1F. sgRNA guides mutant Cas9 to physically inhibit binding of transcription factors in regulatory regions or intronically.

FIG. 2. Overview and workflow of CRISPR/Cas9 construct validation. 2A. Single guide RNAs are designed in silico; 2B. sgRNA oligonucleotides are cloned into the pGS-U6-gRNA vector which contains a human U6 promoter; 2C. double transfection of both vectors into HEK293 cells; 2D. results of gene modification are shown in gel electrophoresis, FIGS. 3 and 4.

FIG. 3. SNCA CRISPR/Cas9 constructs for exon 2. Columns: SNCA 1, SNCA 2, SNCA 3 tested in HEK293 cells. Arrows in lane 2, 4, 6, and 8 point to bands that resulted from cleavage by Cel-1 which allows to detect mismatch changes between DNA strands indicating a double strand break induced insertion/deletion in a pool of cells. 2% AgaroseEX on E-gel iBase Power System, 11 min electrophoresis time. DNA was extracted by GeneArt QE DNA kit.

FIG. 4. MAPT CRISPR/Cas9 constructs for exon 2. Columns: MAPT 1, MAPT 2, MAPT 3 tested in HEK293 cells. Arrows in lane 2, 4, 6, and 8 point to bands that resulted from cleavage by Cel-1 which allows to detect mismatch changes between DNA strands indicating a double strand break induced insertion/deletion in a pool of cells. 2% AgaroseEX on E-gel iBase Power System, 11 min electrophoresis time. DNA was extracted by GeneArt QE DNA kit.

FIG. 5. Schematic overview of testing non-coding regulation regions via luciferase assay. 5A. In silico comparison of evolutionary conserved regions; 5B. Cloning of identified ncECR elements into pGL3 luc+ vector; 5C. transfection into HEK293 or neuroblastoma cells; 5D. Luciferase assay shows regulation of luciferin gene.

FIG. 6. Overview of all ncECR regions in the SNCA locus. Arrows indicate the ncECRs on the genomic locus 4q21. Exons are indicated abbreviated as Ex, D, In, U distinguish regions downstream (D), intronic (In), and upstream (U) of the SNCA gene (UCSC Genome Browser on Human Mar. 2006 Assembly, NCBI36/HG18 (Sterling et al., 2014)).

FIG. 7. Functionally confirmed non-coding regulatory elements in the a-synuclein gene (Sterling et al., 2014). Panels A-C show results of ncECRs upstream (7A), intragenic (7B), and downstream (7C) of the SNCA gene. The X-axis shows the ncECRs, the Y-axis shows the ratio of luciferase and renilla expression as percentage. Bas=pGL3 basic, Con=pGL3 control, prom=pGL3 promoter construct. All red or green box plot elements represent ncECRs that modulate expression significantly. The box plots show the median (horizontal line within box), the 25 and 75% tiles (horizontal borders of box), and the whiskers show the minimal and maximal values. 7C: Luciferase assay results of D6 element cloned into the pGL3 control vector construct. The X-axis shows the D6 and pGL3 control in green and red, respectively, the Y-axis shows the ratio of luciferase and renilla expression as percentage.

FIG. 8. Schematic overview of testing Cas9 inhibition of non-coding regulatory regions via luciferase assay and SNCA expression in vitro. 8A. Single guide RNAs are designed in silico, which is further discussed at Example 2 herein, examples of sgRNA for selected ncECRs D6 and I12 and listed in Table 6; 8B. sgRNA oligonucleotides are cloned into pdCas9/hU6 vector which contains a human U6 promoter and the mutant dead Cas9 protein, second vector is the pGL3 promoter with cloned ncECR shown in FIG. 6; 8C. double transfection of both vectors into HEK293 or neuroblastoma; and 8D. Luciferase assay shows regulation of luciferin gene and expression analysis of SNCA gene.

FIG. 9. Predicted intracellular distribution of alpha-synuclein isoforms. 9A. Alpha-synuclein gene isoform 112, lacking exon 5, has a similar structure as compared to the two published constructs 1-102aa-eGFP (Specht et al., 2005) and 140aa-NES (Kontopoulos et al., 2006) which promote cytoplasmic distribution. Cytoplasmic distribution of alpha-synuclein has shown to be neuroprotective. The Alpha-synuclein gene isoform 112 can be created by double CRISPR editing thus releasing exon 5. 9B. Alpha-synuclein gene isoform 126, lacking exon 3, has some aspects of structure of two published constructs 103-140aa-eGFP (Specht et al., 2005) And 140aa-NES (Kontopoulos et al., 2006) which promote nuclear distribution. Nuclear distribution of alpha-synuclein has been shown to be cytotoxic. aa: amino acid, NES: nuclear export sequence, NLS: nuclear localization signal.

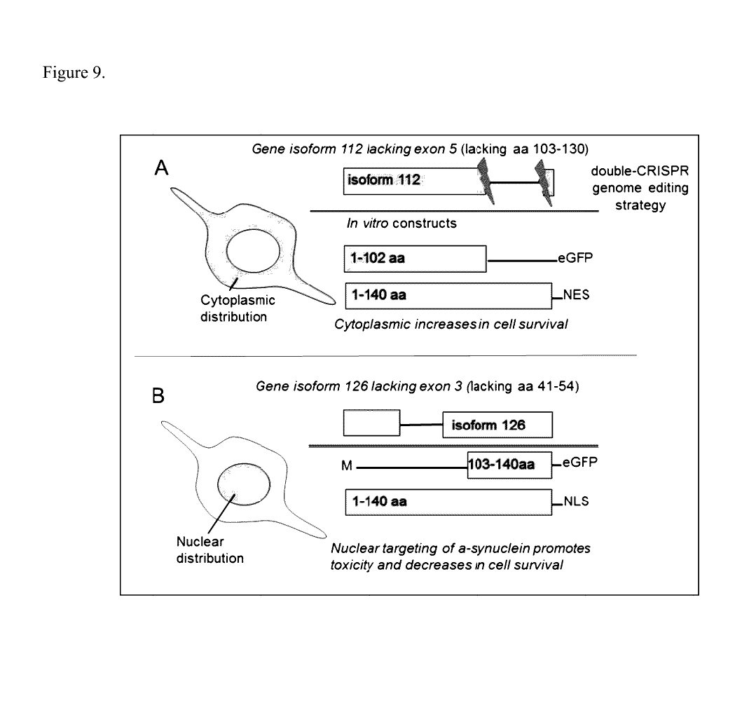

FIG. 10. Options in the treatment of neurological deficits. Different combined delivery and transplantation therapeutic approaches based on disease stage, genetics (e.g. GBA mutations or SNCA risk factors), and additional symptoms (e.g. cognitive decline). In early stages of disease, only viral gene delivery could be sufficient to stop disease progression. In later stages of the disease, gene edited or modified HLA-typed iPS cells, human embryonic stem cells, or patient-derived iPS cells gene modified for SNCA and/or MAPT are transplanted as pre-differentiated cells. Abbreviations: PD=Parkinson's disease, H&Y=Hoehn and Yahr P D disease stage.

FIG. 11. Treatment Options of Ex vivo gene editing and In vivo gene editing. Illustration of ex vivo gene editing in combination with cell transplantation by gene disruption, gene correction, gene modification, or gene repression/activation. Alternativley, direct in vivo gene editing via viral vectors, e.g. AAV, may be used.

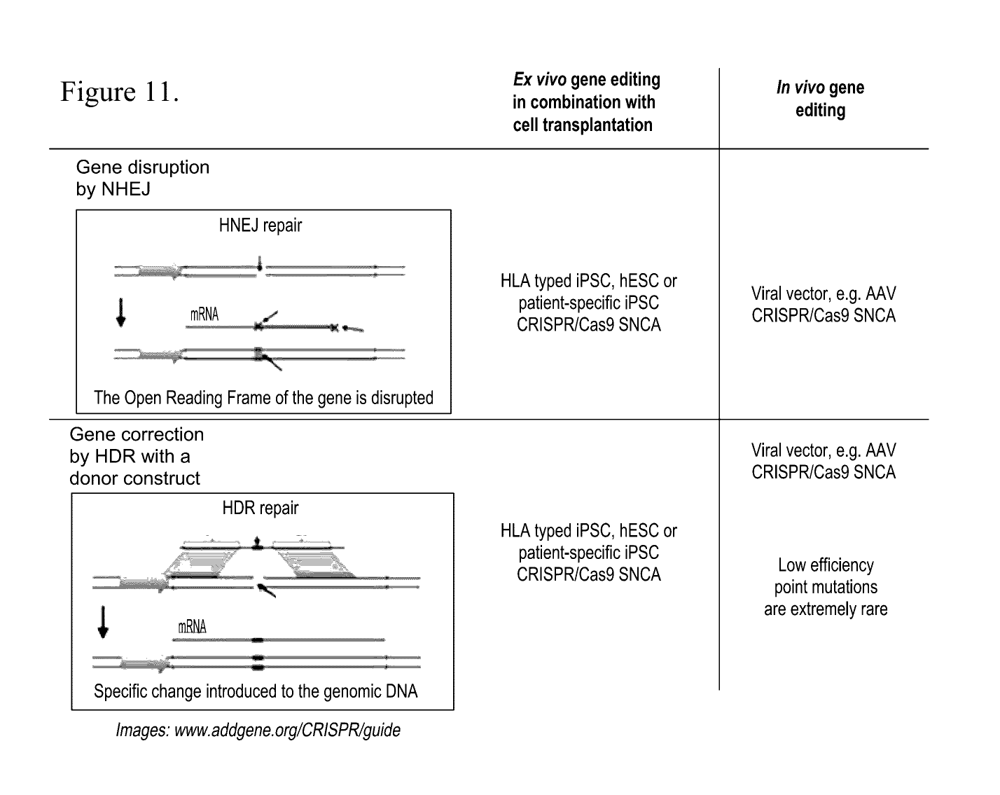

### DETAILED DESCRIPTION OF THE INVENTION

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The tern's can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of “A” will base-pair with “T”.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (Kd) of 10-6 M-1 or lower.

The following definitions and abbreviations are used in the description of the present invention.

A “binding protein” relates to a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, nucleases have DNA-binding, RNA-binding and protein-binding activity.

The term “sequence” relates to a nucleotide sequence of any length, which can be DNA or RNA; can be linear, circular or branched and can be either single-stranded or double stranded.

The term “donor sequence” relates to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “homologous, non-identical sequence” relates to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

The term “identity” relates to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100.

“Sequence similarity” between polynucleotides can be determined by hybridization of polynucleotides under conditions that allow formation of stable duplexes between homologous regions, followed by digestion with single-stranded-specific nuclease(s), and size determination of the digested fragments.

Two nucleic acids, or two polypeptide sequences are substantially “homologous” to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above.

“Recombination” relates to a process of exchange of genetic information between two polynucleotides. For the purposes of this disclosure, “homologous recombination (HR)” relates to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted “nucleases”, e.g. CRIPSR/Cas9, TALEN or ZFN, as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide.

Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween), that are homologous or identical to sequences in the region of interest. In other embodiments, the donor sequence is non-homologous to the first sequence, and is inserted into the genome by non-homologous recombination mechanisms.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule relates to a molecule that is not noanally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule. An exogenous molecule can also be a molecule normally found in another species, for example, a human sequence introduced into an animal's genome.

An “exogenous molecule” can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acidsProteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule relates a molecule in which two or more subunit molecules are linked, preferably covalently. The subunit molecules can be the same chemical type of molecule, or can be different chemical types of molecules. Examples of the first type of fusion molecule include, but are not limited to, fusion proteins and fusion nucleic. Examples of the second type of fusion molecule include, but are not limited to, a fusion between a triplex-foiming nucleic acid and a polypeptide, and a fusion between a minor groove binder and a nucleic acid.

A “gene,” for the purposes of the present disclosure, relates to a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, regulatory sequences such as ribosome binding sites and internal ribosome entry sites, enhancers, silencers, insulators, boundary elements, replication origins, matrix attachment sites and locus control regions.

A “gene regulatory sequence” is a part of a gene that is non-coding for a protein product and can recruit DNA binding protein that can modulate gene expression, either up or downregulation. It can be located upstream or downstream of a gene as well as intragenic.

“Gene expression” or “expression” relates to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression relates to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” relates any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

The CRISPR/Cas9 constructs described herein may be “delivered” or “introduced” into a target cell by any suitable means, including, for example, by injection of mRNA or accordingly nucleic acid, for example, a CDNA, CRNA, or IRNA. See, Hamrnerschmidt et al. (1999) Methods Cell Biol. 59:87-115. Methods of delivering proteins comprising zinc-fingers are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

CRISPR/Cas9 as described herein may also be delivered using “vectors” containing sequences encoding one or more of the CRISPR/Cas9s variations. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more CRISPR/Cas9 encoding sequences. Thus, when one or more pairs of CRISPR/Cas9 are introduced into the cell, the CRISPR/Cas9 may be carried on the same vector or on different vectors.

When multiple vectors are used, each vector may comprise a sequence encoding one or multiple CRISPR/Cas9. Conventional viral and non-viral based gene transfer methods can be used to introduce nucleic acids encoding engineered CRISPR/Cas9 in cells. Such methods can also be used to administer nucleic acids encoding CRISPR/Cas9 to cells in vitro. In certain embodiments, nucleic acids encoding CRISPR/Cas9 are administered for in vivo or ex vivo uses. Non-viral vector delivery systems include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation can also be used for delivery of nucleic acids. Viral vector delivery systems include DNA and RNA viruses, which have either episomal or integrated genomes after delivery to the cell. Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al (2009) Nature Biotechnology vol 27(7) p. 643).

As noted above, the disclosed methods and compositions can be used in any type of cell, progeny, variants and derivatives of animal cells can also be used.

By “Integration” relates to both physical insertion (e.g., into the genome of a host cell) and, in addition, integration by copying of the donor sequence into the host cell genome via the nucleic acid replication processes. Donor sequences can also comprise nucleic acids such as CDNA, CRNA, IRNA, shRNAs, miRNAs, etc. Additional donor sequences of interest may be human genes which encode proteins relevant to disease models.

“Genomic editing” (e.g., inactivation, integration and/or targeted or random mutation) of an animal gene can be achieved, for example, by a single cleavage event, by cleavage followed by non-homologous end joining, by cleavage followed by homology-directed repair mechanisms, by cleavage followed by physical integration of a donor sequence, by cleavage at two sites followed by joining so as to delete the sequence between the two cleavage sites, by targeted recombination of a missense or nonsense codon into the coding region, by targeted recombination of an irrelevant sequence (i.e., a “stuffer” sequence) into the gene or its regulatory region, so as to disrupt the gene or regulatory region, or by targeting recombination of a splice acceptor sequence into an intron to cause mis-splicing of the transcript. See, U.S. Patent Publication Nos. 20030232410; 20050208489; 20050026157; 20050064474; 20060188987; 20060063231; and International Publication WO 07/014,275, the disclosures of which are incorporated by reference in their entireties for all purposes.

“Stem cell” as used herein, refers to an undifferentiated cell that is capable of self-renewal and differentiation into any tissue type of an organism (e.g. neuron, neuroprogenitor cell).

“Brain-related cell” as used herein, refers to a cell of the central nervous system and can comprise of a neural stem cell, neuroprogenitor cell, neuron, glia cell, astrocyte, oligodendrocyte and their progeny.

### Example 1

### Neurodegenerative Targets Disease and Associated Gene

The compositions and methods disclosed have been described with respect to at least one gene, such as SNCA or alpha-synuclein gene (SNCA: synuclein, alpha (non A4 component of amyloid precursor) chromosomal location 4q21.3-q22), and at least one disorder, Parkinson's disease (Online Mendelian Inheritance in Man, OMIM #607060). However, others skilled in the art will appreciate that the methods of gene editing and modification and delivery of the constructs described herein are generally applicable to those neurodegenerative diseases in which over expression or decreased expression of the noimal wildtype sequence or specific genetic variants causes neurodegenerative disease or syndromes The neurodegenerative diseases and genes are listed in Tables 1 and 2.

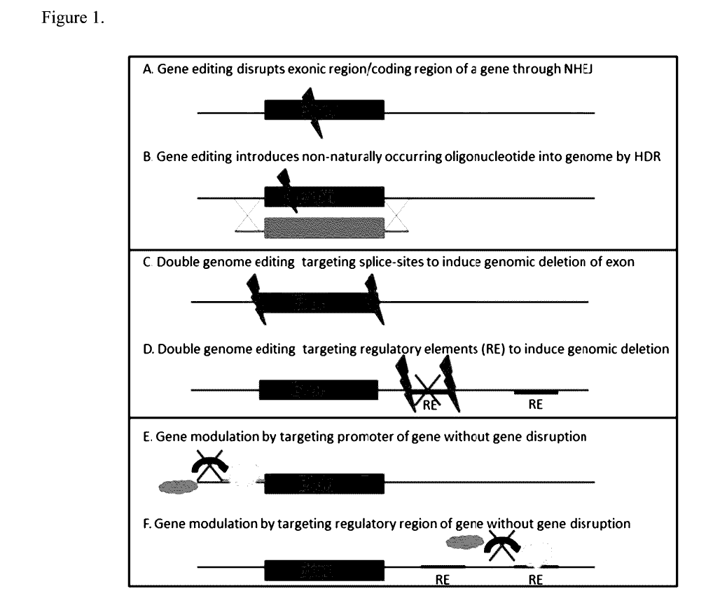

An overview of the different targeting strategies is visualized in FIG. 1. Gene editing or genome editing with engineered nucleases relies on the introduction of double-strand breaks in the DNA by specific enzymes, called nucleases or ‘molecular scissors’. Currently, there are four known such nucleases; zinc-finger nuclease (ZFN), TAL effector nuclease (TALEN), clustered, regularly interspaced, short palindromic repeat (CRIPSR/Cas9) or meganuclease. These nucleases are use alone or in combination, e.g. Cas9 and Fok1. These double strand breaks are not random events, but can be placed very specifically in the genome with guided sequences complementary to the target sequence of the genome.

To further increase the efficiency for the system by multiplexing, additional guide RNAs are designed against the specific SNCA exons 3, 4, or 5, which are listed in Table 4. Multiplexing relates to combining multiple constructs in one transfection by delivering several guide RNAs simultaneously.

### Example 2

### CRISPR sgRNA in Silico Design

Several publicly available bioinformatics tools are used to design oligonucleotides for small guide RNAs.

The in silico design workflow requires input the target sequence (e.g. SNCA coding region, Human Genome Build hg19, length <250 bp) into the CRISPR Design Tool with the following settings: 1) “other region (25-500 nt)” for sequence type and 2) “human (hg19)” for target genome. The CRISPR Design Tool output list of sgRNA designs is ranked according to their “quality scores”. Selection criteria are: 1) high quality guides or/and 2) a variety of sgRNA designs that are far apart from one another and on both strands (+/−) for non-coding ECRs. The candidate designs have to be subsequently assessed for off-target analysis.

To identify the sgRNA sequence for the SNCA gene, the consensus mRNA was used for the SNCA gene, transcript variant 1 (longest isoform, containing all 6 exons) NM_000345.3 (Genome Reference Assembly hg19/GRCh37) and individually searched exons for sgRNA target sequences. The designs with the highest predictive score and least mis-matches using Cas-OFFinder and GGGenome are selected for further validation. Targeting exonic (coding) sequence will introduce random mutations that will lead to novel allelic forms of the gene. These mutations (insertions, deletions) can alter the reading frame of the transcribed RNA and lead to a non-functional protein.

To identify the sgRNA sequence for the MAPT gene, the consensus mRNA was used for the MAPT gene, transcript variant 1 (longest isoform, containing all exons) NM_016835.4 (Genome Reference Assembly hg19/GRCh37) and individually searched exons for sgRNA target sequences. These mutations (insertions, deletions) can alter the reading frame of the transcribed RNA and lead to a non-functional protein. The three designs with the highest predictive score and least mis-matches using Cas-OFFinder and GGGenome are selected for further validation. Targeting exonic (coding) sequence will introduce random mutations that will lead to novel allelic forms of the gene.

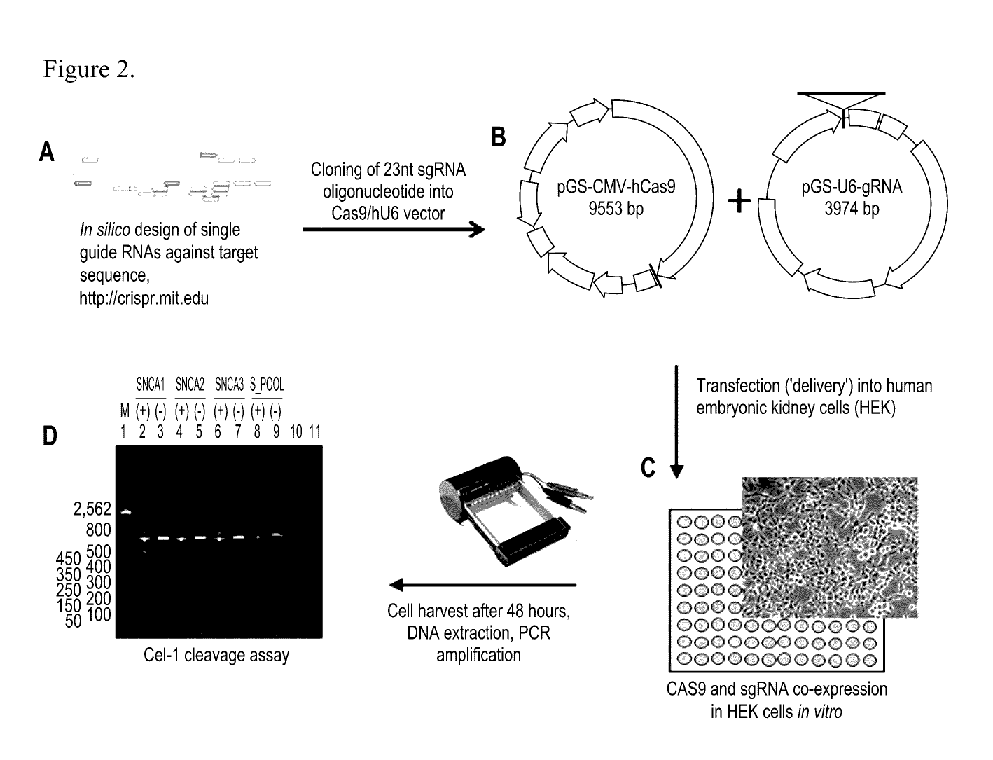

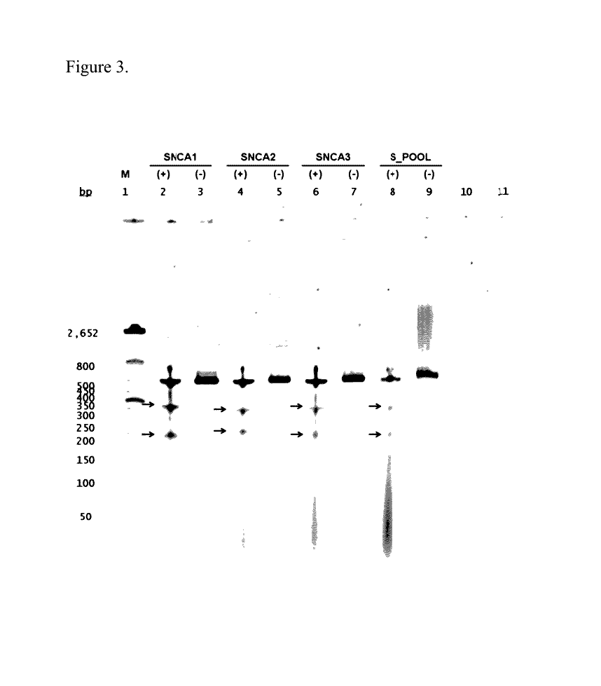

These sgRNA sequences will be experimentally proven and verified. A schematic overview illustrates the workflow (FIG. 2). Table 3 shows SNCA CRISPR guide RNA design in exon 2 for three target guide sequences. FIG. 3 shows the experimental verification of the SNCA CRISPR/Cas9 constructs, U6 guide RNA vector and CMV humanized Cas9 vector, in human HEK293 cells. The HEK293 cells were plated the day before transfection. On the day of transfection the cells were 80-90% confluent and then transfected with 500 ng each of DNA construct with Lipofectamine (Life Technologies). 48 hours later the HEK293 cells were harvested using GeneArt QE DNA kit. DNA was amplified with custom-designed primers to amplify exon 2. The PCR product was subjected to an enzyme Cel-1 that cleaves the DNA where there are heterozygous mutations present. The main PCR amplification band was about 500 bp. The arrows in lane 2, 4, 6, and 8 point to bands that resulted from cleavage by Cel-1 which allows us to detect mismatch changes between DNA strands indicating a double strand break induced insertion/deletion in a pool of cells. Three microliter of PCR reaction was run on 2% AgaroseEX on E-gel iBase Power System.

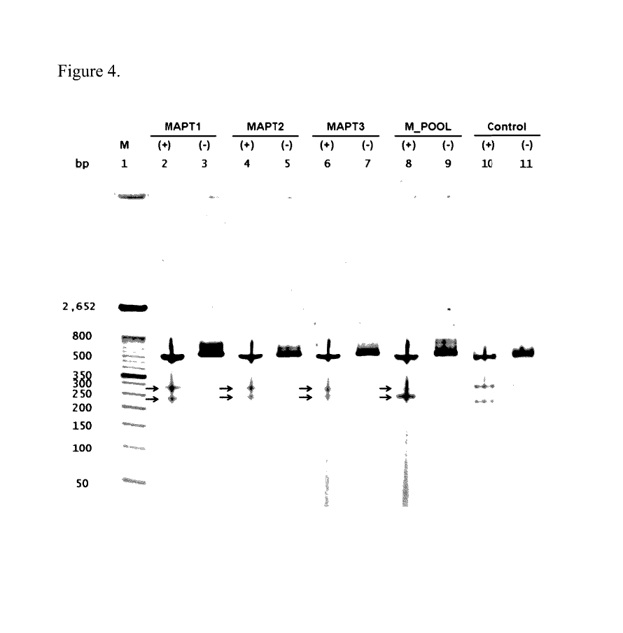

The sgRNA sequences for the MAPT gene will be to experimentally proven and verified in parallel with the SNCA studies. Table 5 shows MAPT CRISPR guide RNA design in exon 2 for three target guide sequences. FIG. 4 shows the experimental verification of the MAPT CRISPR/Cas9 constructs, U6 guide RNA vector and CMV humanized Cas9 vector, in human HEK293 cells. The HEK293 cells were plated the day before transfection. On the day of transfection the cells were 80-90% confluent and then transfected with 500 ng each of DNA constructs with Lipofectamine (Life Technologies). 48 hours later the HEK293 cells were harvested using by GeneArt QE DNA kit. DNA was amplified with custom-designed primers for amplifying exon 2 and PCR product was subjected to an enzyme Cel-1 that cleaves the DNA when there are heterozygous mutations present. The main PCR amplification band was about 500 bp. The arrows in lane 2, 4, 6, and 8 point to bands that resulted from cleavage by Cel-1 which allows to detect mismatch changes between DNA strands indicating a double strand break induced insertion/deletion in a pool of cells. Three microliter of PCR reaction was run on 2% AgaroseEX on E-gel iBase Power System.

### Example 3

### Gene Regulation to Downregulate Alpha-Synuclein

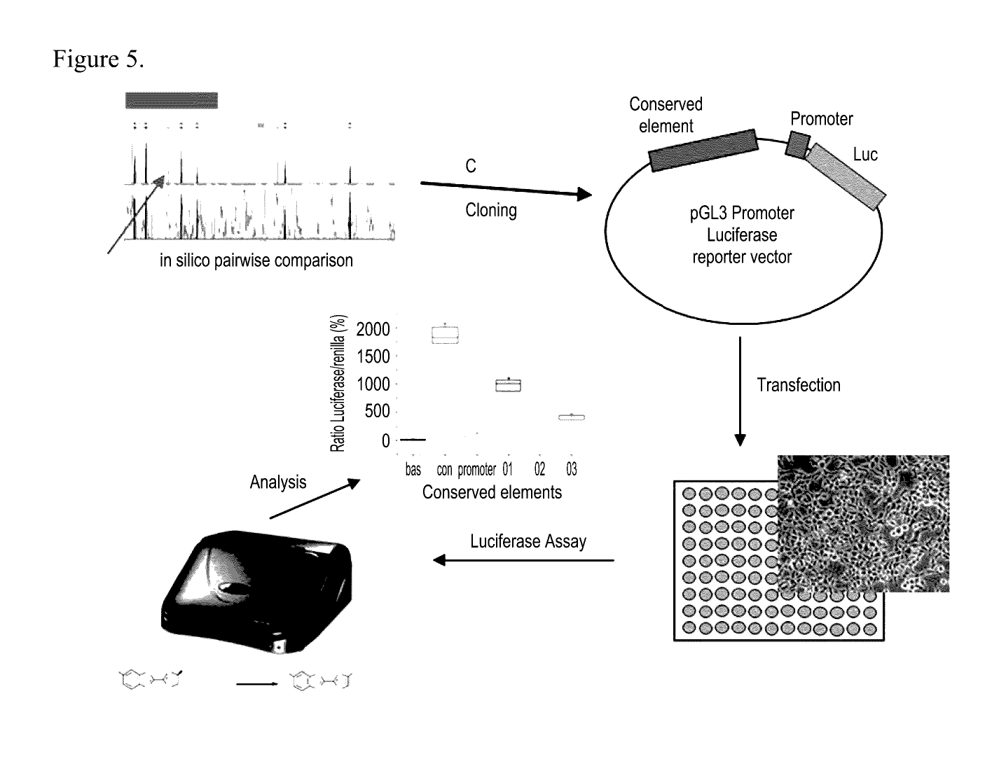

Gene regulation can be achieved by a mutant dCas9 construct, a catalytically inactive programmable RNA-dependent DNA binding protein, fused to VP16 tetramer activation domain or a Krueppel-associated box (KRAB) repressor domain. Twelve regulatory domains were identified within the SNCA gene (Sterling et al., 2014) (FIGS. 5-7) that can be targeted similar to the guide RNAs within the exons of the SNCA and MAPT gene. By mutant CRISPR/dCas9 binding to specific regulatory sites fused to a repressor domain, alpha-synuclein expression can be down regulated.

The catalytically dead Cas-9 (dCas9) lacking endonuclease from type II CRISPR system can control gene expression when co-expressed with sgRNA. The dCas9 generates a DNA recognition complex that does not cleave the DNA, but that can specifically interfere with transcriptional elongation, RNA polymerase binding, and/or transcription factor binding (Jinek et al., 2012; Qi et al., 2013). This system does not alter the genome but modifies gene expression by steric hindrance and is reversible (Kearns et al., 2014; Larson et al., 2013; Qi et al., 2013; Sampson and Weiss, 2014; Xu et al., 2014). The system also does not need the host machinery to function, whereas with genome editing, the cell needs proper function of certain protein and pathway functions e.g. NHEJ or HDR (Xu et al., 2014) FIG. 1, panel C).

Besides the inhibition of gene expression, Cas9 can also be combined with a transactivator and and sgRNAs to induce specific expression of endogenous target genes, which could be a versatile approach for RNA-guided gene activation (Farzadfard et al., 2013).

The dCas9 can be fused to either a CP16 tetramer activation domain (VP64) or a Krueppel-associated box (KRAB) repressor domain. The CRISPR/dCas9 effector system can either be used to modulate or block transcription (CRISPR interference) (Bikard et al., 2013; Kearns et al., 2014; Qi et al., 2013) or activate gene expression using and incorporated effector domain (CRISPR activation) targeting e.g. the promoter region of a gene (Gilbert et al., 2013; Kearns et al., 2014; Konermann et al., 2013; Mali et al., 2013) (FIG. 1, panel C).

Gene Regulation Through Evolutionary Conserved Genomics Regions.

Two main mechanisms are involved in the transcriptional regulation of genes. The first mechanism is gene accessibility, which is regulated through chromatin structure, nucleosome positioning, histone modifications, and DNA methylation. The second factor involves the initiation of transcription through distinct transcription factors such as activators or repressors, and other modulating factors. Transcription factors bind to genomic regions with specific recognition sites.

Identification of 32 Evolutionary Conserved Non-Coding Genomic Regions (ncECRs) within the SNCA Gene

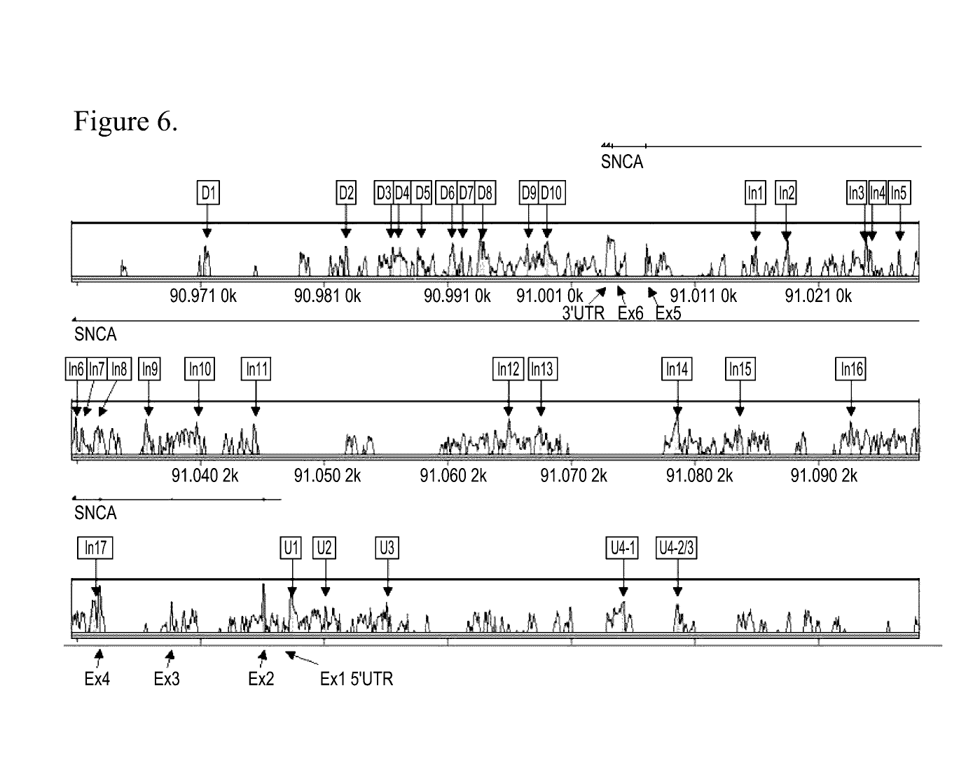

To identify evolutionary conserved non-coding regions, two complementary genome browsers were used (Vista browser (http://pipeline.lb1.gov/cgi-bin/gateway2) and ECR browser (http://ecrbrowser.dcode.org/)) to establish a genetic conservation profile of the SNCA gene by aligning the human SNCA gene with mouse in a pair-wise fashion. Established selection parameters were >100 bp in length and >75% identity (Dubchak et al., 2000; Loots et aL, 2000). In addition to 111.4 kb SNCA genomic region, a 44.5 kb upstream and a 50 kb downstream intergenic region to capture surrounding promoter and regulatory elements (Sterling et al., 2014) (FIG. 6).

32 non-coding evolutionary conversed regions (ncECRs) in the SNCA gene region of 206 kb on chromosome 4q21 (Chr.4: 90, 961056-91, 167082) were identified by pair-wise comparison between human and mouse (FIG. 6). In comparison to similar screens, where conserved regions range from 8-45 elements (Grice et al., 2005; Liu and Francke, 2006; Sabherwal et al., 2007), a similar number of elements was found that show a high evolutionary conservation. (Sterling et al., 2014).

The Promega pGL3 luciferase reporter vectors and the neuroblastoma cell line SK-N-SH were used as a tool to determine the SNCA regulatory elements. NcECRs identified through the comparative analysis were cloned upstream of a SV-40 promoter in the pGL3 promoter construct, transfected in neuroblastoma cells and assayed in a dual luciferase assay by measuring firefly luciferase and renilla luciferase as an internal control to normalize the firefly signal (FIG. 5) (Sterling et al., 2014).

Cells were transfected at 90-95% confluency. All transfections were performed in quadruplicates and each experiment was repeated three times. For the luciferase assay, the Dual-Luciferase® Reporter (DLR™) Assay System (Promega) was used, in which activities of firefly and Renilla luciferases can be measured sequentially in one sample (Sterling et al., 2014).

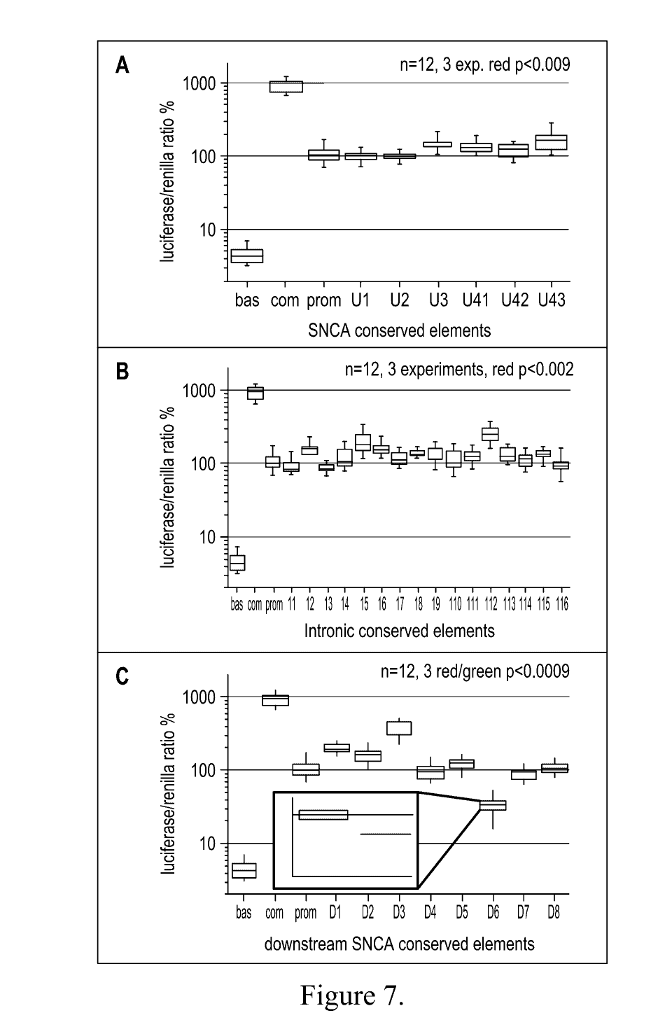

Overall, 12 of 32 constructs exhibited either an enhancement or reduction of the expression of the reporter gene (FIG. 2). Three elements upstream of the SNCA gene displayed an approx. 1.5 fold (p<0.009) increase in expression (U3, U4-1, U4-3). Of the intronic regions, three showed a 1.5 fold increase (12, 16, 18) and two others caused a 2 and 2.5 fold increase (I5, I12) in expression (p<0.002). Two elements downstream of the SNCA gene showed 1.5 fold (D1, D2) and 2.5 fold increase (D3; p<0.0009). One element downstream of SNCA had a reduced expression of the reporter gene of 0.35 fold (D6; p<0.0009) of normal activity that was also continued in a pGL3 control vector (FIG. 2C, insert). The D6 element reduced the expression of the pGL3 control construct by ˜50%, confirming that this element represents a strong repressor. These data provide experimental evidence that a significant proportion of the ncECRs show a regulatory function in the luciferase reporter assay (FIG. 7) (Sterling et al., 2014).

The NACP-Rep1 polymorphism of the SNCA promoter, a mixed dinucleotide repeat, that increases the expression of alpha-synuclein when the long allele is present. A mutant Cas9 repressor/CRISPR guide RNA against this region will facilitate downregulation of gene expression of alpha-synuclein (FIG. 1C).

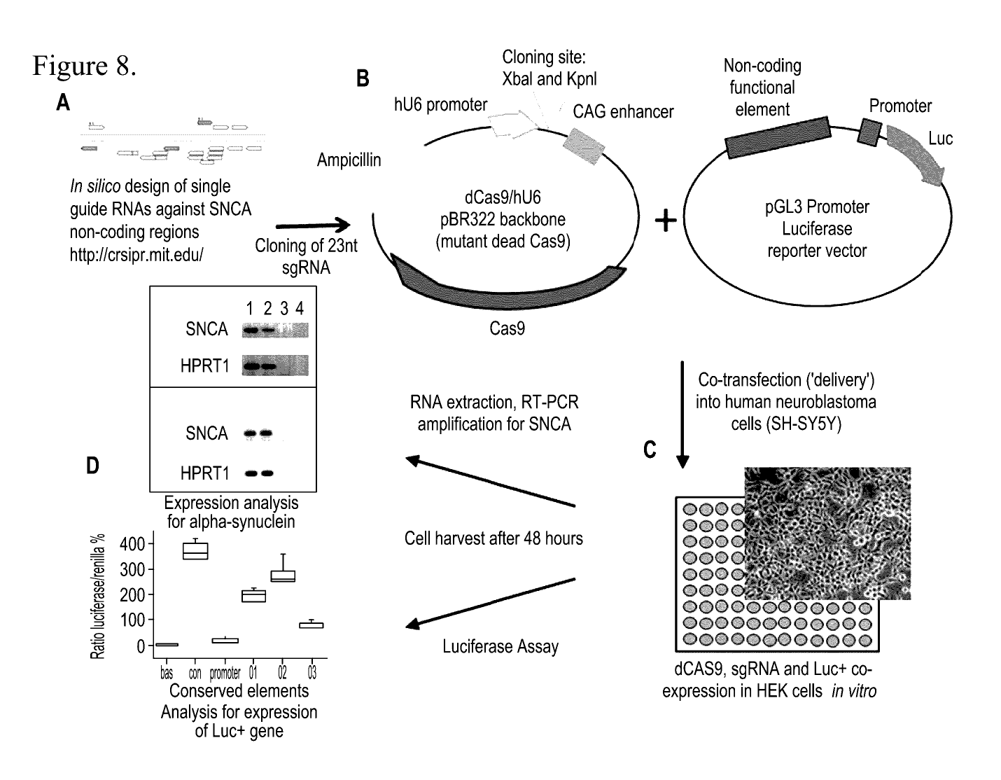

With the identification of regulatory elements in the SNCA gene, CRISPR guide RNAs will be designed against these regulatory regions to modestly downregulate the expression of alpha-synuclein. Gene downregulation will be achieved by a mutant Cas9 construct that can be fused to a Krueppel-associated box (KRAB) repressor domain. The advantage of the system is that the regulation is modest versus a complete knockdown and at the same time abolishing its normal function (FIG. 8).

Practice of the methods, as well as preparation and use of the compositions disclosed herein employ, unless otherwise indicated, conventional techniques in molecular biology, biochemistry, gene or chromatin structure analysis, computational biology, cell culture, recombinant DNA methods and related methods or techniques within the skill of the art. These techniques and principles are fully explained in the literature.

Although the disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### Example 4

### Predicted Intracellular Distribution of Alpha-Synuclein Isoforms

The full length alpha-synuclein has 140 amino acids. However, there are several smaller alpha synuclein proteins that lack certain exon(s) and therefore are shorter in nature, isoform 112, isoform 126, and isoform 96. SNCA gene isoform 112, lacking exon 5, has a similar structure as compared to two published constructs 1-102aa-eGFP (Specht et al., 2005) and 140aa-NES (Kontopoulos et al., 2006), which promote cytoplasmic distribution. Cytoplasmic distribution of alpha-synuclein has shown to be neuroprotective. The Alpha-synuclein gene isoform 112 can be created by double CRISPR editing thus releasing exon 5. Using a double gene editing approach as discussed in FIG. 1C or 1D will result in specific splice forms of the alpha-synuclein protein and potentially be neuroprotective.

### Prophetic Example 5

### Virus Delivery System for In Vivolin Human Gene Editing for Neurodegeneration Disease Progression

The delivery vehicle that will be utilized for Cas9/CRISPR delivery will be an adeno-associated virus (AAV) (Bartus et al., 2013; Bartus et al., 2011b; Marks et al., 2010) or lentiviral (Palfi et al., 2014) vector that have been previously used for gene delivery in human brain for PD. Both viruses have been used in clinical trials in human for brain delivery and have been proven in phase I/II clinical trials to be safe and well tolerated (Bartus et al., 2014; Kaufmann et al., 2013). Other viral delivery options for the gene editing technology proposed in this application will be considered when available and if deemed to be more advantageous over the aforementioned approaches.

Over the last several years, a total of 9 gene therapy clinical trials have been performed for the treatment of PD. Even though the clinical benefits were small or clinical endpoints were not reached, these clinical trials were necessary and important to demonstrate that gene therapy in the central nervous system is not producing an untoward risk or harm after administration of different viral vector delivery constructs. The gene therapy trials have also paved the road for new clinical trial designs, patient selection, and outcome measures based on treatment specific differences with gene therapy in the CNS (Bartus et al., 2014).

Adeno-associated viral vectors are excellent vehicles to transfer genes into the nervous system due to their property to transduce also post-mitotic cells, their ability to be grown to very high titers (up to 10¹³ virion particles per ml), and their relatively large insert capacity (insert capacity of ˜7.5 kb). Adenoviral vectors can express the transgene(s) for a long time in the CNS in vivo and in cell culture such as neurons and glia (Southgate et al., 2008).

Lentiviral vectors have shown to have a low immunogenicity, can transduce neurons, and can carry large inserts which allows for the introduction of multiple sgRNAs against several locations of one gene or multiple genes concurrently (Cockrell and Kafri, 2007).

The CRISPR gene editing or gene regulation approach will be delivered in the brain via stereotactic surgery into the substantia nigra pars compacta (Kells et al., 2012; Salegio et al., 2012). Depending on the state of the art at the time of entering into clinical trials, stereotactic administration into the substantia nigra or other basal ganglia will be determined.

Gene therapy delivery systems discussed for in vivo gene editing application in human. These do not form part of the instant application but are provided for information only.

Gene editing in patient-derived stem cells or human embryonic stem cells followed by neuronal transplantation as a therapeutic approach to prevent neurodegeneration and disease progression.

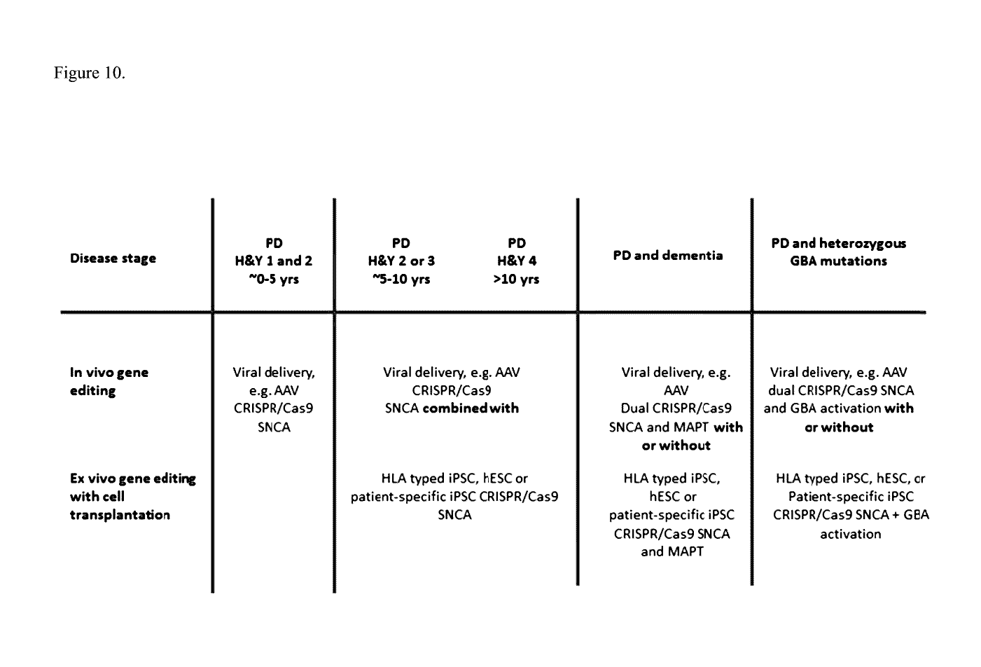

For more advanced stages of neurodegeneration in PD or related neurodegenerative disease different approaches as proposed (FIG. 10). Besides the direct treatment with AAV CRISPR constructs including sgRNA targeting SNCA or MAPT, human stem cells from different sources, either human embryonic stem cells (hESC) or HLA-typed induced pluripotent stem cells (iPSC) or individual patient-derived pluripotent stem cell clones are being gene modified with CRISPR/sgRNA SNCA to specifically lower the expression of alpha-synuclein in a controlled fashion. Correctly targeted stem cell clones are then differentiated into neuronal cultures (neuroprogenitor cells) and subsequently transplanted into the basal ganglia in the CNS.

Fetal Transplant Trials in Parkinson's Disease.

Estimates from pre-clinical models suggest that one dopaminergic neurons in the substantia nigra in the brainstem can innervate up to 75,000 neurons in the neostriatum, on the other hand one striatal neuron can be under the influence of 100-200 dopaminergic neurons (Matsuda et al., 2009). This suggests that here must be an enormous energy requirement for dopaminergic neurons, but there is also major redundancy of the system which could explain why motor symptoms of PD only present when dopaminergic demise reaches a critical threshold of loss of 70-80% of striatal nerve terminals and 50-60% of the nuclei in the SNc (Bernheimer et al., 1973; Riederer and Wuketich, 1976).

If dopaminergic neurons could be replenished and transplanted neuronal cells would survive, re-innervate the lesions in the midbrain and improve symptoms, the disease could potentially be cured. This has been the working hypothesis for cellular replacement in PD for the last 35 years and is a major hope for patients to overcome the disease (Bjorklund et al., 2003; Freed et al., 2011).

The first report on cell replacement in a parkinsonian pre-clinical model was published in 1979, when rats were transplanted with fetal stem cells which survived, had axonal outgrowth and motor abnormalities were reduced (Perlow et al., 1979). The first fetal transplant surgery in human was performed in 1987 and since then an estimated 300 PD patients have received implantation of human fetal tissue from 6-9 week old aborted fetuses (Clarkson, 2001). Initial open-label studies have been promising and provided proof of principle that grafted DA neurons can improve motor symptoms of PD, however, the interpretation of the functional improvements seen in these open-label studies has been questioned because placebo effects and/or natural history of some mild forms of PD (Freed et al., 1993; Freed et al., 1992a; Freed et al., 1990a; Freed et al., 1990b; Freed et al., 1992b; Lindvall et al., 1990; Lindvall et al., 1989).

Long-term outcome of fetal transplants were first reported in 2008 with several publications reporting alpha-synuclein positive Lewy body pathology in grafts of autopsies from brain donors, showing that the disease process continues and the healthy graft also degenerates over time (Kordower et al., 2008; Li et al., 2008; Mendez et al., 2008).

The gene editing approach of alpha-synuclein modulation and repression will ‘shield’ or protect the dopaminergic neurons in the brain as well as the transplanted neurons described above. Similarly this approach can be utilized for protein aggregation of tau or other proteins aggregating in the cell.

### Example 6

### Virus Delivery System

Viral vectors: The delivery vehicle that will be utilized for Cas9/CRISPR delivery will be an adeno-associated virus (AAV) (Bartus et al., 2013; Bartus et al., 2011b; Marks et al., 2010) or lentiviral (Palfi et al., 2014) vector that have been previously used for gene delivery in human brain for PD. Both viruses have been used in clinical trials in human for brain delivery and have been proven in phase I/II clinical trials to be safe and well tolerated (Bartus et al., 2014; Kaufmann et al., 2013). Other viral delivery options for the gene editing technology proposed in this application will be considered when available and if deemed to be more advantageous over the aforementioned approaches.

AAV vectors: Adeno-associated viral vectors are excellent vehicles to transfer genes into the nervous system due to their property to transduce also post-mitotic cells, their ability to be grown to very high titers (up to 10¹³ virion particles per ml), and their relatively large insert capacity (insert capacity of ˜7.5 kb). Adenoviral vectors can express the transgene(s) for a long time in the CNS in vivo and in cell culture such as neurons and glia (Southgate et al., 2008).

Lentiviral vectors: Lentiviral vectors have shown to have a low immunogenicity, can transduce neurons, and can carry large inserts which allows for the introduction of multiple sgRNAs against several locations of one gene or multiple genes concurrently (Cockrell and Kafri, 2007).

Gene editing design: The CRISPR/Cas9 gene will be cloned into the delivery vector such as AAV or lentivirus together with the designed sgRNA against selected regions of the SNCA or MAPT gene. The sgRNA can comprise of any one or combination of the sgRNA designs in this application, either exonic, intronic, promoter region, or regulatory regions.

Stereotactic surgery: The CRISPR gene editing or gene regulation approach will be delivered in the brain via stereotactic surgery into the substantia nigra pars compacta (Kells et al., 2012; Salegio et al., 2012). Depending on the state of the art at the time of entering into clinical trials, stereotactic administration into the substantia nigra or other basal ganglia will be determined. The clinical trial design will be based on prior designs of neuromodulating therapies (Bartus et al., 2011a; Marks et al., 2010; Marks et al., 2008).

Patient population: The patient populations envisioned for this treatment of gene editing of the SNCA genes are patients with Parkinson's disease in early disease stages, such as Hoehn and Yahr 1 and 2, when the symptoms are unilateral or bilateral, but no gait instability or balance problems are present. Patients with early signs of cognitive decline or mild cognitive impairment can be candidates for the MAPT in vivo gene editing.

### Example 7

### Gene Editing in Patient-Derived Stem Cells or Human Embryonic Stem Cells Followed by Neuronal Transplantation

Replenishment of dopaminergic neurons and the survival of transplanted neuronal cells, it is expected that re-innervation of the lesions in the midbrain and improvement of symptoms, would be treated. This has been the working hypothesis for cellular replacement in PD. (Bjorklund et al., 2003; Freed et al., 2011).

Stem cell derivation and characterization: Different stem cell lines can be used for this approach: (1) Well-characterized human embryonic stem cell (ESC) lines; (2) Human derived induced pluripotent stem cells (iPSCs) from the patient; or (3) Matched HLA-type iPSCs. Human ESCs or matched HLA-type iPSCs can be obtained from a cell bank whereas patient's own iPSCs can be grown and reprogrammed in animal free conditions and under good manufacturing practices (GMP). Stem cells are derived and they passed quality control of gene expression for pluripotency markers, markers for all three germ layers, and potential to differentiate in target tissue of interest (e.g. neurons). Cells are vigorously tested for genetic changes during in vitro culture.

CRISPR/Cas9 cloning: After tissue derivation, the CRISPR/Cas9 gene will be cloned into the delivery vector separate or together with the designed sgRNA against selected regions of the SNCA or MAPT gene. The sgRNA can comprise of any one or combination of the sgRNA designs in this application, either exonic, intronic, promoter region, or regulatory regions. Designs for direct viral delivery or ex vivo editing use the same sgRNA sequences. The vector design can vary.

Ex vivo editing: Human stem cells from different sources (hESCs, iPSCs) are being gene modified with CRISPR/sgRNA SNCA to specifically lower the expression of alpha-synuclein in a controlled fashion. Gene editing vectors are delivered via electroporation or nucleofection. Stem cells recover from nucelofection of one passage before they are subcloned and single clones are tested for gene modification by various genetic amplification methods (T7 endonuclease treatment, gene sequencing or droplet PCR). Correctly targeted stem cell clones are then differentiated into neuronal cultures (neuroprogenitor cells) either as embryoid body/neural rosette cultures (Ebert et al., 2013; Shelley et al., 2014; Swistowski et al., 2009), small molecule-induced direct differentiation (Ganat et al., 2012; Kriks et al., 2011) (Cooper et al., 2010; Hargus et al., 2010) and subsequently transplanted into the basal ganglia in the CNS (Clarkson, 2001; Freeman et al., 1995; Kordower et al., 1998; Olanow et al., 2001; Olanow et al., 2003).

Patient population: Ex vivo gene modification with subsequence transplantation is proposed from move advance stages of Parkinson's disease, disease stage Hoehn and Yahr 2 and higher.

### Example 8

### Viral Gene Delivery and Gene Edited Cell Transplantation

A third alternative approach is the combination of viral gene delivery and transplantation of gene edited cells to preserve the existing neurons and replenish with the new neurons that are less likely develop disease due to its gene modification which promotes neuroprotection.

### Tables

TABLE 1
Neurodegenerative diseases
A non-exhaustive list of neurodegenerative diseases that can be
targeted in the brain with the proposed invention is presented in
Table 1. The examples of diseases listed can be targeted with
this method when a specific gene is known to cause the disease.

| | Phenotype |
|---|---|
| Disease; Disease Symbol | MIM Number |
| Dementia with Lewy bodies; DLB | #127750 |
| Parkinson's disease | #168600 |
| Parkinson's disease 1, autosomal dominant; PARK1 | #168601 |
| Parkinson's disease, juvenile, type 2; PARK2 | #600116 |
| Parkinson's disease, juvenile, type 6; PARK6 | #605909 |
| Parkinson disease 7, autosomal recessive early-onset; | #606324 |
| PARK7 | |
| Parkinson's disease 8, autosomal dominant; PARK8 | #607060 |
| Frontotemporal dementia, Pick complex included; FTD | #600274 |
| Kufor-Rakeb Syndrome; KRS | #606693 |
| Parkinson disease 14, autosomal recessive; PARK14 | #612953 |
| Heterogeneous phenotype: dystonia-parkinsonism, | #256600 |
| dementia, frontotemporal atrophy, w/wo brain | |
| iron accumulation | |
| Neurodegeneration with brain iron accumulation 2A; | #256600 |
| NBIA2A | |
| Parkinsonian-Pyramidal Syndrome; PARK15 | #260300 |
| Parkinson's disease 17; PARK17 | #614203 |
| Parkinson's disease 18; PARK18 | #614251 |
| Parkinson disease 19, Juvenile-onset; PARK19 | #615528 |
| Parkinson's disease 20; PARK20 | #615530 |
| Dystonia-Parkinsonism, X-Linked; DYT3 | #314250 |
| Dopamine transporter deficiency syndrome; DTDS | #613135 |
| Gaucher disease; GD1 | #230800 |
| Tay-Sachs disease; TSD | #272800 |
| Niemann-Pick disease, type A | #257200 |
| Niemann-Pick disease, type B | #607616 |
| Alzheimer disease; AD | #104300 |
| Alzheimer disease, type 3; AD3 | #607822 |
| Pick disease | #172700 |
| Multiple system atrophy; MSA1 | #146500 |
| Dystonia, dopa-responsive; DRD, DYT5 | #128230 |
| Perry syndrome, parkinsonism with alveolar | #168605 |
| hypoventilation and mental depression | |
| Dystonia-Parkinsonism, X-linked, Lubag disease, | #314250 |

The database Online Mendelian Inheritance of Man (MIM) Ahttp://www.ncbi.nlm.nih.gov/omim)>, the contents of which are incorporated herein by reference, describes genetic causation and association factors for a range of disease states and conditions, indexing the genes according to a “Gene/Locus MIM Number” and the disease states and conditions according to a “Phenotype MIM Number”. Table 1 below provides non-limiting examples of genetically linked adult-onset neurodegeneration diseases listed in the Online MIM. For further details of each condition, including the chromosomal locations and names of the genes, as well as literature references and discussion of the underlying research, please refer to Table 2 below and the OMIM website.

TABLE 2
A non-exhaustive list of examples of genes
A non-exhaustive list of examples of genes that can be targeted for Parkinsonian
disorders, memory function and related disorders recessive disorder is provided below.

| Symbol | | | | Inheritance | |
|---|---|---|---|---|---|
| for the | | | | (AD = autosomal | Gene/Locus |
| Disease, | HUGO gene name, | | | dominant; | Number or |
| HUGO | symbol, locus, reference | Gene | | AR = autosomal | other MIM |
| Gene | sequence | locus | Disorder | recessive) | Reference |
| PARK1, | Synuclein, alpha (non A4 | 4q21-22 | Parkinson's disease, | AD | #168601 |
| SNCA | component of amyloid | | Dementia with Lewy | | |
| | precursor), SNC4, chr4q21-22, | | bodies | | |
| | NM_000345.3 | | | | |
| MAPT | microtubule-associated | | Frontotemporal | AD | #157140 |
| | protein tau, 17q21.31 | | dementia, with or | | |
| | | | without parkinsonism, | | |
| | | | Alzheimer disease | | |
| PSEN1 | Presinilin 1, 14q24.2 | 14q24.2 | Frontotemporal | AD | #104311 |
| | | | dementia, Alzheimer | | |
| | | | disease | | |
| PARK 2, | Parkin RBR E3 ubiquitin | 6q25.2-q27 | Parkinson's disease, | AR | #602544 |
| PARKIN | protein ligase, PARK2, | | juvenile, type 2 | | |
| | chr6q25.2-p27 | | | | |
| | NM_004562.2 | | | | |
| PARK4, | Synuclein, alpha (non A4 | 4q21-22 | Parkinson's disease, | AD | #605543 |
| SNCA | component of amyloid | | Dementia with Lewy | | |
| | precursor) | | bodies | | |
| PARK6, | PTEN induced putative | 1p35-p36 | Parkinson's disease, | AR | #608309 |
| PINK1 | kinase 1, PINK1, chr1p35-36, | | juvenile, type 6 | | |
| | NM_032409.2 | | | | |
| PARK7, | Daisuke-Junko 1, DJ-1 | 1p36 | Parkinson's disease 7, | AR | #602533 |
| DJ-1 | chr1p36 | | autosomal recessive | | |
| | NM_001123377.1 | | early-onset | | |
| PARK8, | Leucine-rich repeat kinase | 12q12 | Parkinson's disease, | AD | #609007 |
| LRRK2 | 2, LRRK2, chr12q12, | | Parkinson's disease | | |
| | NM_198578.3 | | dementia, Dementia | | |
| | | | with Parkinson's | | |
| | | | disease | | |
| PARK9, | ATPase type 13A2, | 1p36 | Kufor-Rakeb | AR | #610513 |
| ATP13A2 | ATP13A2, | | syndrome | | |
| | chr1p36, NM_022089.3 | | | | |
| PARK14, | Phospholipase A2, group VI | 22q13.1 | Dystonia- | AR | *603604 |
| PLA2G6 | (cytosolic, calcium- | | parkinsonism, | | |
| | independent), PLA2G6, | | dementia, | | |
| | chr22q13.1, NM_003560.2 | | frontotemporal | | |
| | | | atrophy, w/wo brain | | |
| | | | iron accumulation, | | |
| | | | pallido-pyramidal | | |
| | | | syndrome | | |
| PARK15, | F-box protein 7, FBXO7, | 22q12-q13 | Pallido-pyramidal | AR | #605648 |
| FBX07 | chr22q12-q13, | | syndrome | | |
| | NM_012179.3 | | | | |
| PARK17, | Vacuolar protein sorting 35 | 16q11.2 | Parkinson's disease 17 | AD | *601501 |
| VPS35 | homolog (S. cerevisiae), | | | | |
| | VPS35, chr16q11.2, | | | | |
| | NM_018206.4 | | | | |
| PARK18, | Eukaryotic translation | 3q27.1 | Parkinson's disease 18 | AD | *600495 |
| EIF4G1 | initiation factor 4 gamma, 1, | | | | |
| | EIF4G1, chr3q27.1, | | | | |
| | NM_001291157.1 | | | | |
| PARK19, | DnaJ (Hsp40) homolog, | 1p31.3 | Neurodegenerative | AR | #608375 |
| DNAJC6 | subfamily C, member 6, | | disorder including | | |
| | DNAJC6 | | parkinsonism and | | |
| | chr1p31.3, | | additional features | | |
| | NM_001256864.1 | | | | |
| PARK20, | Synaptojanin 1, SYNJ1 | 21q22.11 | Parkinson disease 20, | AR | #604297 |
| SYNJ-1 | chr21q22.11, NM_203446.2 | | early-onset | | |
| DNAJC13 | DnaJ (Hsp40) homolog, | 3q22.1 | Parkinson's disease, | AD | *614334 |
| | subfamily C, member 13, | | parkinsonism, | | |
| | DNAJC13, chr3q22.1, | | DNAJ/HSP40 | | |
| | NM_015268.3 | | HOMOLOG, | | |
| | | | SUBFAMILY C, | | |
| | | | MEMBER 13 | | |
| GCH-1 | GTP cyclohydrolase 1, | 14q22.1- | GTP | AD | *600225 |
| | GCH1, | 14q22.2 | CYCLOHYDROLASE | | |
| | Chr14q22.1-22.2, | | I, Dopa-responsive | | |
| | NM_000161.2 | | dystonia, parkinsonism | | |
| DCTN1 | Dynactin 1, DCTN1, | 2p13 | Dynactin 1, Perry | AD | *601143 |
| | chr2p13, NM_004082.4 | | syndrome, | | |
| | | | parkinsonism | | |
| DYT3, | TAF1 RNA polymerase II, | Xq13.1 | Severe progressive | X-linked | #313650 |
| TAF1 | TATA box binding protein | | torsion dystonia | | |
| | (TBP)-associated factor, | | followed by | | |
| | 250 kDa, TAF1, chrXq13.1, | | parkinsonism | | |
| | NM_004606.4 | | | | |
| SLC6A3 | Solute carrier family 6 | 5p15.33 | Dopamine transporter | AR | *126455 |
| | (neurotransmitter | | deficiency syndrome | | |
| | transporter), member 3, | | (DTDS) | | |
| | SLC6A3chr5p15.33, | | | | |
| | NM_001044.4 | | | | |
| GBA | Glucosidase, beta, acid, | 1q22 | Parkinson's disease | AR | #606463 |
| | GBA, chr1q22, | | | | |
| | NM_000157.3 | | | | |
| HEXA | Beta-hexosaminidase A, | 15q23 | Tay-Sachs disease | AR | *606869 |
| | HEXA, 15q23, NM_000520 | | | | |
| SMPD1 | Sphingomyelin | 11p15.4 | Niemann Pick Type | AR | #607608 |
| | phosphodiesterase 1, acid | | A/B | | |
| | lysosomal, SMPD1, | | | | |
| | 11p15.4, NM_000543 | | | | |
| POLG | POLYMERASE, DNA, | 15q26.1 | Complex neurological | AR | *174763 |
| | GAMMA, POLG, 15q26.1, | | phenotype including | | |
| | NM_001126131 | | Dopa-responsive | | |
| | | | Parkinsonism | | |
| SNCB | Synuclein, beta, SNCB, | 5q35.2 | Alzheimer disease | AD | #602569 |
| | 5q35.2, NM_001001502 | | | | |

TABLE 3
SNCA CRISPR guide RNA design in exon 2
SNCA CRISPR Design
SpCas9 from Streptococcus pyogenes: 5′-NGG-3′
Genome Reference Assembly: Homo sapiens (hg19/GRCh37), February 2009
SNCA Gene Reference Sequence: Homo sapiens synuclein (SNCA), transcript variant 1, mRNA
(NM_000345.3)

| | | | | | GC |
|---|---|---|---|---|---|
| SEQ ID | Name | Target Sequence (5′→3′) | Strand | Genomic Location | Content |
| SEQ ID NO: 1 | SNCA_E2_1 | GTAAAGGAATTCATTAGCCATGG | − | chr4: 90756815- | 39% |
| | | | | 90756837 | |
| SEQ ID NO: 2 | SNCA_E2_2 | GCCATGGATGTATTCATGAAAGG | − | chr4: 90756799- | 43% |
| | | | | 90756821 | |
| SEQ ID NO: 3 | SNCA_E2_3 | TCCTTTCATGAATACATCCATGG | + | chr4: 90756798- | 39% |
| | | | | 90756820 | |

TABLE 4
SNCA CRISPR guide RNA design in exon 3, 4, 5
SNCA CRISPR Design
SpCas9 from Streptococcus pyogenes: 5′-NGG-3′
Genome Reference Assembly: Homo sapiens (hg19/GRCh37), February 2009
SNCA Gene Reference Sequence: Homo sapiens synuclein (SNCA), transcript variant 1, mRNA
(NM_000345.3)

| | | | | | GC |
|---|---|---|---|---|---|
| SEQ ID | Name | Target Sequence (5′→3′) | Strand | Genomic Location | Content |
| SEQ ID NO: 4 | SNCA_E3_1 | ACCAAGGAGGGAGTGGTGCATGG | − | chr4: 90749305-90749327 | 60% |
| SEQ ID NO: 5 | SNCA_E3_3 | ACCATGCACCACTCCCTCCTTGG | + | chr4: 90749304-90749326 | 60% |
| SEQ ID NO: 6 | SNCA_E4_1 | CTTTGTCAAAAAGGACCAGTTGG | − | chr4: 90743402-90743424 | 40% |
| SEQ ID NO: 7 | SNCA_E4_2 | GAGCAAGTGACAAATGTTGGAGG | − | chr4: 90743500-90743522 | 45% |
| SEQ ID NO: 8 | SNCA_E5_1 | GCCTCATTGTCAGGATCCACAGG | + | chr4: 90650364-90650386 | 55% |
| SEQ ID NO: 9 | SNCA_E5_3 | GCCTGTGGATCCTGACAATGAGG | − | chr4: 90650365-90650387 | 55% |

TABLE 5
MAPT CRISPR guide RNA design in exon 2
MAPT CRISPR Design
SpCas9 from Streptococcus pyogenes: 5′-NGG-3′
Genome Reference Assembly: Homo sapiens (hg19/GRCh37), February 2009
MAPT Gene: microtubule-associated protein tau (MAPT), transcript variant 1, mRNA
(NM_016835.4)

| | | | | | | GC |
|---|---|---|---|---|---|---|
| SEQ ID | Identifier | Target Sequence (5′→3′) | Strand | Genomic Location | Exon | Content |
| SEQ ID NO: 10 | MAPT_E2_1 | CACGCTGGGACGTACGGGTTGGG | + | chr17: 44039743- | 2 | 70% |
| | | | | 44039765 | | |
| SEQ ID NO: 11 | MAPT_E2_2 | AAGATCACGCTGGGACGTACGGG | + | chr17: 44039738- | 2 | 61% |
| | | | | 44039760 | | |
| SEQ ID NO: 12 | MAPT_E2_5 | ATCACTTCGAACTCCTGGCGGGG | − | chr17: 44039713- | 2 | 61% |
| | | | | 44039735 | | |

TABLE 6
sgRNA designs for non-coding evolutionary conserved regions in SNCA locus
6A. NcECR SNCA D6 sgRNA designs
Chr4: 90636868 + 90637447
Genome Reference Assembly: Homo sapiens (hg19/GRCh37), February 2009
Genomic region covers 582 base pairs

| | | | | | GC |
|---|---|---|---|---|---|
| SEQ ID | Identifier | Target Sequence (5′→3′) | Genomic Location | Strand | Content |
| SEQ ID NO: 13 | SNCA_D6_1 | TGTGAAAGACCGTAAGTTGCTGG | chr4: 90636884-90636906 | + | 45% |
| SEQ ID NO: 14 | SNCA_D6_2 | GGATCAAGACAACTTGGCCATGG | chr4: 90637178-90637200 | + | 50% |
| SEQ ID NO: 15 | SNCA_D6_3 | AAGAGGTGAGTTCTAATCATTGG | chr4: 90637114-90637136 | − | 35% |
| SEQ ID NO: 16 | SNCA_D6_4 | CTTGATGGCAGTCACTTGAAAGG | chr4: 90637246-90637268 | + | 45% |
| SEQ ID NO: 17 | SNCA_D6_5 | ATGGTGTCCTGAGATGACTGAGG | chr4: 90637056-90637078 | + | 50% |

6B. NcECR SNCA I12 sgRNA designs
Chr4: 90721247 + 90721915
Genome Reference Assembly: Homo sapiens (hg19/GRCh37), February 2009
Genomic region covers 669 base pairs

| | | | | | GC |
|---|---|---|---|---|---|
| SEQ ID | Identifier | Target Sequence (5′→3′) | Genomic Location | Strand | Content |
| SEQ ID NO: 18 | SNCA_I12_1 | GTCAAGTTTACTATCTTACGTGG | chr4: 90721433-90721455 | + | 35% |
| SEQ ID NO: 19 | SNCA_I12_2 | TTTCACTTACGTACTATGAAAGG | chr4: 90721479-90721501 | − | 30% |
| SEQ ID NO: 20 | SNCA_I12_3 | GAATTGTACTGACTACACCACGG | chr4: 90721881-90721903 | + | 40% |
| SEQ ID NO: 21 | SNCA_I12_4 | TACTCATATTACAGTCAGTCTGG | chr4: 90721588-90721610 | − | 35% |
| SEQ ID NO: 22 | SNCA_I12_5 | ATCCTTGTATAAACCCCACAAGG | chr4: 90721634-90721656 | + | 40% |

TABLE 7
SNCA promoter region sgRNA designs
Genome Reference Assembly: Homo sapiens (hg19/GRCh37), February 2009;
Genomic region comprises of 996 base pairs on Chr.4: 90756741-90757736

| SEQ ID | Identifier | sgRNA Designs | Genomic Location | Strand |
|---|---|---|---|---|
| SEQ ID NO: 23 | SNCA_Promoter_1 | TAGCCACCTAACCACGGATTAGG | chr4: 90757040-90757062 | + |
| SEQ ID NO: 24 | SNCA_Promoter_2 | GGTTAACAAGTGCTGGCGCGGGG | chr4: 90757456-90757478 | − |
| SEQ ID NO: 25 | SNCA_Promoter_3 | GGGTTAACAAGTGCTGGCGCGGG | chr4: 90757457-90757479 | − |
| SEQ ID NO: 26 | SNCA_Promoter_4 | CATCCTAATCCGTGGTTAGGTGG | chr4: 90757043-90757065 | − |
| SEQ ID NO: 27 | SNCA_Promoter_5 | CGGGTTAACAAGTGCTGGCGCGG | chr4: 90757458-90757480 | − |
| SEQ ID NO: 28 | SNCA_Promoter_6 | AGTTGGATGCTCACGCTCCATGG | chr4: 90757598-90757620 | + |
| SEQ ID NO: 29 | SNCA_Promoter_7 | TGGAGCATCCTCGCGTTTCCCGG | chr4: 90757618-90757640 | + |
| SEQ ID NO: 30 | SNCA_Promoter_8 | GAGCATCCTCGCGTTTCCCGGGG | chr4: 90757620-90757642 | + |
| SEQ ID NO: 31 | SNCA_Promoter_9 | GCTTTTCCCCGGGAAACGCGAGG | chr4: 90757626-90757648 | − |
| SEQ ID NO: 32 | SNCA_Promoter_10 | ATTCATCCTAATCCGTGGTTAGG | chr4: 90757046-9075706 | − |
| SEQ ID NO: 33 | SNCA_Promoter_11 | AGAACGCCCCCTCGGGTGGCTGG | chr4: 90757414-90757436 | − |
| SEQ ID NO: 34 | SNCA_Promoter_13 | TGCTGGCGCGGGGTCCGCTAGGG | chr4: 90757446-90757468 | − |
| SEQ ID NO: 35 | SNCA_Promoter_14 | TGGCGCGGGGTCCGCTAGGGTGG | chr4: 90757443-90757465 | − |
| SEQ ID NO: 36 | SNCA_Promoter_15 | AGAGGATTCATCCTAATCCGTGG | chr4: 90757051-90757073 | − |
| SEQ ID NO: 37 | SNCA_Promoter_16 | GGTAACGGGTTAACAAGTGCTGG | chr4: 90757463-90757485 | − |
| SEQ ID NO: 38 | SNCA_Promoter_17 | GCAGGCGCCGCTCACACTCGCGG | chr4: 90757373-9075739 | + |
| SEQ ID NO: 39 | SNCA_Promoter_18 | CAGGCGCCGCTCACACTCGCGGG | chr4: 90757374-90757396 | + |
| SEQ ID NO: 40 | SNCA_Promoter_19 | TCCCGGGGAAAAGCGGATCCCGG | chr4: 90757635-90757657 | + |
| SEQ ID NO: 41 | SNCA_Promoter_20 | GGAGCATCCTCGCGTTTCCCGGG | chr4: 90757619-90757641 | + |
| SEQ ID NO: 42 | SNCA_Promoter_21 | ACTGTTACCCTTTAGACCCCGGG | chr4: 90757137-90757159 | − |
| SEQ ID NO: 43 | SNCA_Promoter_22 | TTTAAATTCCCGGGGTCTAAAGG | chr4: 90757129-90757151 | + |
| SEQ ID NO: 44 | SNCA_Promoter_23 | GCTGAGAACGCCCCCTCGGGTGG | chr4: 90757418-90757440 | − |
| SEQ ID NO: 45 | SNCA_Promoter_24 | CTAACTGCTCACTCGGGGTGTGG | chr4: 90757266-90757288 | + |
| SEQ ID NO: 46 | SNCA_Promoter_25 | AGACGGCCCGCGAGTGTGAGCGG | chr4: 90757380-90757402 | − |
| SEQ ID NO: 47 | SNCA_Promoter_26 | TTAAATTCCCGGGGTCTAAAGGG | chr4: 90757130-90757152 | + |
| SEQ ID NO: 48 | SNCA_Promoter_27 | CAGAACTAACTGCTCACTCGGGG | chr4: 90757261-90757283 | + |
| SEQ ID NO: 49 | SNCA_Promoter_28 | TCGGGGTGTGGTTCAAACTCAGG | chr4: 90757278-90757300 | + |
| SEQ ID NO: 50 | SNCA_Promoter_29 | TAAAGTAAGCAAGCTGCGTTTGG | chr4: 90757528-90757550 | − |
| SEQ ID NO: 51 | SNCA_Promoter_30 | GGAAACGCGAGGATGCTCCATGG | chr4: 90757615-90757637 | − |
| SEQ ID NO: 52 | SNCA_Promoter_31 | TCGCGTTTCCCGGGGAAAAGCGG | chr4: 90757628-90757650 | + |
| SEQ ID NO: 53 | SNCA_Promoter_32 | TACTGTTACCCTTTAGACCCCGG | chr4: 90757138-90757160 | − |
| SEQ ID NO: 54 | SNCA_Promoter_33 | ACCCCGCGCCAGCCACCCGAGGG | chr4: 90757406-90757428 | + |
| SEQ ID NO: 55 | SNCA_Promoter_34 | AAAGTCTAGCCACCTAACCACGG | chr4: 90757034-90757056 | + |
| SEQ ID NO: 56 | SNCA_Promoter_35 | GTGTACACTCATTTAACCATTGG | chr4: 90756947-90756969 | + |
| SEQ ID NO: 57 | SNCA_Promoter_36 | TGAATTCCTTTACACCACACTGG | chr4: 90756825-90756847 | + |
| SEQ ID NO: 58 | SNCA_Promoter_37 | GGAGGCTGAGAACGCCCCCTCGG | chr4: 90757422-90757444 | − |
| SEQ ID NO: 59 | SNCA_Promoter_38 | GAGGCTGAGAACGCCCCCTCGGG | chr4: 90757421-90757443 | − |
| SEQ ID NO: 60 | SNCA_Promoter_39 | CCCCTCGGGTGGCTGGCGCGGGG | chr4: 90757407-90757429 | − |
| SEQ ID NO: 61 | SNCA_Promoter_40 | GGATTAGAACCATCACACTTGGG | chr4: 90757312-90757334 | − |
| SEQ ID NO: 62 | SNCA_Promoter_41 | AATCTGTCTGCCCGCTCTCTTGG | chr4: 90757561-90757583 | − |

TABLE 8
SNCA intron 4 and 5 region sgRNA designs
SNCA intron 4 guide RNAs
Genome Reference Assembly: Homo sapiens (hg19/GRCh37), February 2009.

| SEQ ID | Identifier | sgRNA Designs | Genomic Location | Strand |
|---|---|---|---|---|
| SEQ ID NO: 63 | SNCA_Intron 4_1 | TTGCCGCAATGTTTCCCCGGAGG | Chr4: 90650595-90650617 | − |
| SEQ ID NO: 64 | SNCA_Intron 4_2 | GCACACCTTAATATTACTACTGG | Chr4: 90650553-90650575 | + |
| SEQ ID NO: 65 | SNCA_Intron 4_3 | GGGAAACATTGCGGCAACATGGG | Chr4: 90650601-90650623 | + |
| SEQ ID NO: 66 | SNCA_Intron 4_4 | GGGGAAACATTGCGGCAACATGG | Chr4: 90650600-90650622 | + |
| SEQ ID NO: 67 | SNCA_Intron 4_5 | TGTGCCATTTTCAAGATCCGTGG | Chr4: 90650535-90650557 | − |
| SEQ ID NO: 68 | SNCA_Intron 4_6 | TAAACTCCACAATGCCTCCGGGG | Chr4: 90650581-90650603 | + |
| SEQ ID NO: 69 | SNCA_Intron 4_7 | ATGTTGCCGCAATGTTTCCCCGG | Chr4: 90650598-90650620 | − |
| SEQ ID NO: 70 | SNCA_Intron 4_8 | ATGCCTCCGGGGAAACATTGCGG | Chr4: 90650592-90650614 | + |
| SEQ ID NO: 71 | SNCA_Intron 4_1 | TTGGCCACGGATCTTGAAAATGG | Chr4: 90650531-90650553 | + |
| SEQ ID NO: 72 | SNCA_Intron 4_10 | AATCTTACATATAGGGATGTTGG | Chr4: 90650512-90650534 | + |
| SEQ ID NO: 73 | SNCA_Intron 4_11 | TCTAAACTCCACAATGCCTCCGG | Chr4: 90650579-90650601 | + |
| SEQ ID NO: 74 | SNCA_Intron 4_12 | ATGTTTCCCCGGAGGCATTGTGG | Chr4: 90650587-90650609 | − |
| SEQ ID NO: 75 | SNCA_Intron 4_13 | CTAAACTCCACAATGCCTCCGGG | Chr4: 90650580-90650602 | + |
| SEQ ID NO: 76 | SNCA_Intron 4_14 | GAATGCCAGTAGTAATATTAAGG | Chr4: 90650558-90650580 | − |
| SEQ ID NO: 77 | SNCA_Intron 4_15 | ACATATAGGGATGTTGGCCACGG | Chr4: 90650518-90650540 | + |
| 1. | 2. | 3. | 4. | 5. |
| 6. | 7. | 8. | 9. | 10. |

SNCA intron 5 guide RNAs
Genome Reference Assembly: Homo sapiens (hg19/GRCh37), February 2009

| SEQ ID | Identifier | sgRNA Designs | Genomic Location | Strand |
|---|---|---|---|---|
| SEQ ID NO: 78 | SNCA_Intron 5_1 | CTCTCTACATGCTCATTACGTGG | Chr4: 90650228-90650250 | − |
| SEQ ID NO: 79 | SNCA_Intron 5_2 | CTTACTACTTGACCCTTTACAGG | Chr4: 90650077-90650099 | − |
| SEQ ID NO: 80 | SNCA_Intron 5_3 | AGTAAGTATATTAAGCTATGTGG | Chr4: 90650094-90650116 | + |
| SEQ ID NO: 81 | SNCA_Intron 5_4 | GTCATCCCATTGGACATGTATGG | Chr4: 90649933-90649955 | + |
| SEQ ID NO: 82 | SNCA_Intron 5_5 | TTTACTATGCGTCATCCCATTGG | Chr4: 90649923-90649945 | + |
| SEQ ID NO: 83 | SNCA_Intron 5_6 | TTTTGCCATACATGTCCAATGGG | Chr4: 90649938-90649960 | − |
| SEQ ID NO: 84 | SNCA_Intron 5_7 | TTTTTGCCATACATGTCCAATGG | Chr4: 90649939-90649961 | − |
| SEQ ID NO: 85 | SNCA_Intron 5_8 | GGAAATCAATGGAATGCCAAAGG | Chr4: 90649973-90649995 | − |
| SEQ ID NO: 86 | SNCA_Intron 5_9 | TGGAAAGTTAGGGAAATCAATGG | Chr4: 90649984-90650006 | − |
| SEQ ID NO: 87 | SNCA_Intron 5_10 | ATCAATGGAATGCCAAAGGAAGG | Chr4: 90649969-90649991 | − |

### SEQUENCE LISTING

The sequences referred to in the above description by Identification Numbers are as follows:

| SEQ ID | Target Sequence (5′→3′) |
|---|---|
| SEQ ID NO: 1 | GTAAAGGAATTCATTAGCCATGG |
| SEQ ID NO: 2 | GCCATGGATGTATTCATGAAAGG |
| SEQ ID NO: 3 | TCCTTTCATGAATACATCCATGG |
| SEQ ID NO: 4 | ACCAAGGAGGGAGTGGTGCATGG |
| SEQ ID NO: 5 | ACCATGCACCACTCCCTCCTTGG |
| SEQ ID NO: 6 | CTTTGTCAAAAAGGACCAGTTGG |
| SEQ ID NO: 7 | GAGCAAGTGACAAATGTTGGAGG |
| SEQ ID NO: 8 | GCCTCATTGTCAGGATCCACAGG |
| SEQ ID NO: 9 | GCCTGTGGATCCTGACAATGAGG |
| SEQ ID NO: 10 | CACGCTGGGACGTACGGGTTGGG |
| SEQ ID NO: 11 | AAGATCACGCTGGGACGTACGGG |
| SEQ ID NO: 12 | ATCACTTCGAACTCCTGGCGGGG |
| SEQ ID NO: 13 | TGTGAAAGACCGTAAGTTGCTGG |
| SEQ ID NO: 14 | GGATCAAGACAACTTGGCCATGG |
| SEQ ID NO: 15 | AAGAGGTGAGTTCTAATCATTGG |
| SEQ ID NO: 16 | CTTGATGGCAGTCACTTGAAAGG |
| SEQ ID NO: 17 | ATGGTGTCCTGAGATGACTGAGG |
| SEQ ID NO: 18 | GTCAAGTTTACTATCTTACGTGG |
| SEQ ID NO: 19 | TTTCACTTACGTACTATGAAAGG |
| SEQ ID NO: 20 | GAATTGTACTGACTACACCACGG |
| SEQ ID NO: 21 | TACTCATATTACAGTCAGTCTGG |
| SEQ ID NO: 22 | ATCCTTGTATAAACCCCACAAGG |
| SEQ ID NO: 23 | TAGCCACCTAACCACGGATTAGG |
| SEQ ID NO: 24 | GGTTAACAAGTGCTGGCGCGGGG |
| SEQ ID NO: 25 | GGGTTAACAAGTGCTGGCGCGGG |
| SEQ ID NO: 26 | CATCCTAATCCGTGGTTAGGTGG |
| SEQ ID NO: 27 | CGGGTTAACAAGTGCTGGCGCGG |
| SEQ ID NO: 28 | AGTTGGATGCTCACGCTCCATGG |
| SEQ ID NO: 29 | TGGAGCATCCTCGCGTTTCCCGG |
| SEQ ID NO: 30 | GAGCATCCTCGCGTTTCCCGGGG |
| SEQ ID NO: 31 | GCTTTTCCCCGGGAAACGCGAGG |
| SEQ ID NO: 32 | ATTCATCCTAATCCGTGGTTAGG |
| SEQ ID NO: 33 | AGAACGCCCCCTCGGGTGGCTGG |
| SEQ ID NO: 34 | TGCTGGCGCGGGGTCCGCTAGGG |
| SEQ ID NO: 35 | TGGCGCGGGGTCCGCTAGGGTGG |
| SEQ ID NO: 36 | AGAGGATTCATCCTAATCCGTGG |
| SEQ ID NO: 37 | GGTAACGGGTTAACAAGTGCTGG |
| SEQ ID NO: 38 | GCAGGCGCCGCTCACACTCGCGG |
| SEQ ID NO: 39 | CAGGCGCCGCTCACACTCGCGGG |
| SEQ ID NO: 40 | TCCCGGGGAAAAGCGGATCCCGG |
| SEQ ID NO: 41 | GGAGCATCCTCGCGTTTCCCGGG |
| SEQ ID NO: 42 | ACTGTTACCCTTTAGACCCCGGG |
| SEQ ID NO: 43 | TTTAAATTCCCGGGGTCTAAAGG |
| SEQ ID NO: 44 | GCTGAGAACGCCCCCTCGGGTGG |
| SEQ ID NO: 45 | CTAACTGCTCACTCGGGGTGTGG |
| SEQ ID NO: 46 | AGACGGCCCGCGAGTGTGAGCGG |
| SEQ ID NO: 47 | TTAAATTCCCGGGGTCTAAAGGG |
| SEQ ID NO: 48 | CAGAACTAACTGCTCACTCGGGG |
| SEQ ID NO: 49 | TCGGGGTGTGGTTCAAACTCAGG |
| SEQ ID NO: 50 | TAAAGTAAGCAAGCTGCGTTTGG |
| SEQ ID NO: 51 | GGAAACGCGAGGATGCTCCATGG |
| SEQ ID NO: 52 | TCGCGTTTCCCGGGGAAAAGCGG |
| SEQ ID NO: 53 | TACTGTTACCCTTTAGACCCCGG |
| SEQ ID NO: 54 | ACCCCGCGCCAGCCACCCGAGGG |
| SEQ ID NO: 55 | AAAGTCTAGCCACCTAACCACGG |
| SEQ ID NO: 56 | GTGTACACTCATTTAACCATTGG |
| SEQ ID NO: 57 | TGAATTCCTTTACACCACACTGG |
| SEQ ID NO: 58 | GGAGGCTGAGAACGCCCCCTCGG |
| SEQ ID NO: 59 | GAGGCTGAGAACGCCCCCTCGGG |
| SEQ ID NO: 60 | CCCCTCGGGTGGCTGGCGCGGGG |
| SEQ ID NO: 61 | GGATTAGAACCATCACACTTGGG |
| SEQ ID NO: 62 | AATCTGTCTGCCCGCTCTCTTGG |
| SEQ ID NO: 63 | TTGCCGCAATGTTTCCCCGGAGG |
| SEQ ID NO: 64 | GCACACCTTAATATTACTACTGG |
| SEQ ID NO: 65 | GGGAAACATTGCGGCAACATGGG |
| SEQ ID NO: 66 | GGGGAAACATTGCGGCAACATGG |
| SEQ ID NO: 67 | TGTGCCATTTTCAAGATCCGTGG |
| SEQ ID NO: 68 | TAAACTCCACAATGCCTCCGGGG |
| SEQ ID NO: 69 | ATGTTGCCGCAATGTTTCCCCGG |
| SEQ ID NO: 70 | ATGCCTCCGGGGAAACATTGCGG |
| SEQ ID NO: 71 | TTGGCCACGGATCTTGAAAATGG |
| SEQ ID NO: 72 | AATCTTACATATAGGGATGTTGG |
| SEQ ID NO: 73 | TCTAAACTCCACAATGCCTCCGG |
| SEQ ID NO: 74 | ATGTTTCCCCGGAGGCATTGTGG |
| SEQ ID NO: 75 | CTAAACTCCACAATGCCTCCGGG |
| SEQ ID NO: 76 | GAATGCCAGTAGTAATATTAAGG |
| SEQ ID NO: 77 | ACATATAGGGATGTTGGCCACGG |
| SEQ ID NO: 78 | CTCTCTACATGCTCATTACGTGG |
| SEQ ID NO: 79 | CTTACTACTTGACCCTTTACAGG |
| SEQ ID NO: 80 | AGTAAGTATATTAAGCTATGTGG |
| SEQ ID NO: 81 | GTCATCCCATTGGACATGTATGG |
| SEQ ID NO: 82 | TTTACTATGCGTCATCCCATTGG |
| SEQ ID NO: 83 | TTTTGCCATACATGTCCAATGGG |
| SEQ ID NO: 84 | TTTTTGCCATACATGTCCAATGG |
| SEQ ID NO: 85 | GGAAATCAATGGAATGCCAAAGG |
| SEQ ID NO: 86 | TGGAAAGTTAGGGAAATCAATGG |
| SEQ ID NO: 87 | ATCAATGGAATGCCAAAGGAAGG |

### BIBLIOGRAPHY

The following references are hereby incorporated into the present application by reference.
(1) Ahituv, N., et al. (2004). Human molecular genetics 13 Spec No 2, R261-266.
(2) Appel-Cresswell, et al. (2013). Movement disorders: official journal of the Movement Disorder Society 28, 811-813.
(3) Bartus, R. T., et al. (2013). Neurology 80, 1698-1701.
(4) Bartus, R. T., et al. (2011a). Neurobiology of disease 44, 38-52.
(5) Bartus, R. T., et al. (2011b). Movement disorders: official journal of the Movement Disorder Society 26, 27-36.
(6) Bartus, R. T., et al. (2014). Molecular therapy: the journal of the American Society of Gene Therapy 22, 487-497.
(7) Beneke, S., et al. (2007). Nucleic Acids Res 35, 7456-7465.
(8) Bernheimer, H., et al. (1973). Journal of the neurological sciences 20, 415-455.
(9) Beyer, K. (2006). Acta neuropathologica 112, 237-251.
(10) Beyer, K., et al. (2008). Neurogenetics 9, 15-23.
(11) Beyer, K., et al. (2006). Neuroreport 17, 1327-1330.
(12) Beyer, K., et al. (2004). Neuropathology and applied neurobiology 30, 601-607.
(13) Bikard, D., et al. (2013). Nucleic Acids Res 41, 7429-7437.
(14) Bird, C. P., et al. (2006). Curr Opin Genet Dev 16, 559-564.
(15) Bjorklund, A., et al. (2003). The Lancet Neurology 2, 437-445.
(16) Campion, D., et al. (1995). Genomics 26, 254-257.
(17) Chartier-Harlin, M. C., et al. (2004). Lancet 364, 1167-1169.
(18) Chen, H., et al. (2014). The Journal of biological chemistry 289, 13284-13294.
(19) Chen, Z. J., et al. (2005). Biochemical and biophysical research communications 329, 646-652.
(20) Chiba-Falek, O., et al. (2005). American journal of human genetics 76, 478-492.
(21) Chiba-Falek, O., et al. (2001). Human molecular genetics 10, 3101-3109.
(22) Chiba-Falek, O., et al. (2003). Human genetics 113, 426-431.
(23) Clarkson, E. D. (2001). Drugs & aging 18, 773-785.
(24) Clough, R. L., et al. (2011). Journal of neurochemistry 117, 275-285.
(25) Clough, R. L., et al. (2009). Journal of neurochemistry 110, 1479-1490.
(26) Clough, R. L., et al. (2007). FASEB journal: official publication of the Federation of American Societies for Experimental Biology 21, 596-607.
(27) Cockrell, A. S., et al. (2007). Molecular biotechnology 36, 184-204.
(28) Cooper, O., et al. (2010). Molecular and cellular neurosciences 45, 258-266.
(29) Cronin, K. D., et al. (2009). Human molecular genetics 18, 3274-3285.
(30) Deng, H., et al. (2014). Ageing research reviews 15, 161-176.
(31) DiFiglia, M., et al. (2007). Proceedings of the National Academy of Sciences of the United States of America 104, 17204-17209.
(32) Ding, Q., et al. (2014). Circ Res 115, 488-492.
(33) Dorsey, E. R., et al. (2007). Neurology 68, 384-386.
(34) Dubchak, I., et al. (2000). Genome Res 10, 1304-1306.
(35) Ebert, A. D., et al. (2013). Stem cell research 10, 417-427.
(36) Farrer, M., et al. (2001). Hum Mol Genet 10, 1847-1851.
(37) Farzadfard, F., et al. (2013). ACS synthetic biology 2, 604-613.
(38) Fauser, F., et al. (2014). The Plant journal: for cell and molecular biology 79, 348-359.
(39) Ferres-Coy, A., et al. (2013). Transl Psychiatry 3, e211.
(40) Forman, M. S., et al. (2005). Neuron 47, 479-482.
(41) Freed, C. R., et al. (1993). Advances in neurology 60, 721-728.
(42) Freed, C. R., et al. (1992a). The New England journal of medicine 327, 1549-1555.
(43) Freed, C. R., et al. (1990a). Archives of neurology 47, 505-512.
(44) Freed, C. R., et al. (1990b). Progress in brain research 82, 715-721.
(45) Freed, C. R., et al. (1992b). Neurochemistry international 20 Suppl, 321S-327S.
(46) Freed, C. R., et al. (2011). Neurotherapeutics: the journal of the American Society for
Experimental NeuroTherapeutics 8, 549-561.
(47) Freeman, T. B., et al. (1995). Annals of neurology 38, 379-388.
(48) Fuchs, J., et al. (2007a). Neurology 68, 916-922.
(49) Fuchs, J., et al. (2007b). Faseb J.
(50) Ganat, Y. M., et al. (2012). The Journal of clinical investigation 122, 2928-2939.
(51) Gelb, D. J., et al. (1999). Arch Neurol 56, 33-39.
(52) Gilbert, L. A., et al. (2013). Cell 154, 442-451.
(53) Gorbatyuk, O. S., et al. (2010). Mol Ther 18, 1450-1457.
(54) Grice, E. A., et al. (2005). Hum Mol Genet 14, 3837-3845.
(55) Hadjigeorgiou, G. M., et al. (2006). Mov Disord 21, 534-539.
(56) Hargus, G., et al. (2010). Proceedings of the National Academy of Sciences of the United States of America 107, 15921-15926.
(57) Heintze, J., et al. (2013). Frontiers in genetics 4, 193.
(58) Holzmann, C., et al. (2003). Journal of neural transmission 110, 67-76.
(59) Hong, M., et al. (1998). Science 282, 1914-1917.
(60) Hutton, M., et al. (1998). Nature 393, 702-705.
(61) Ibanez, P., et al. (2004). Lancet 364, 1169-1171.
(62) Jinek, M., et al. (2012). Science 337, 816-821.
(63) Kaufmann, K. B., et al. (2013). EMBO molecular medicine 5, 1642-1661.
(64) Kearns, N. A., et al. (2014). Development 141, 219-223.
(65) Kells, A. P., et al. (2012). Neurobiology of disease 48, 228-235.
(66) Khodr, C. E., et al. (2014). Brain research 1550, 47-60.
(67) Kiely, A. P., et al. (2013). Acta Neuropathol 125, 753-769.
(68) Klein, C., et al. (2007). Neurology.
(69) Klein, C., et al. (2012). Cold Spring Harb Perspect Med 2, a008888.
(70) Konermann, S., et al. (2013). Nature 500, 472-476.
(71) Kontopoulos, E., et al. (2006). Hum Mol Genet.
(72) Kordower, J. H., et al. (2008). Nat Med 14, 504-506.
(73) Kordower, J. H., et al. (1998). Movement disorders: official journal of the Movement Disorder Society 13, 383-393.
(74) Kriks, S., et al. (2011). Nature.
(75) Kruger, R., et al. (1998). Nat Genet 18, 106-108.
(76) Kruger, R., et al. (1999). Ann Neurol 45, 611-617.
(77) Langston, J. W. (2006). Ann Neurol 59, 591-596.
(78) Larson, M. H., et al. (2013). Nature protocols 8, 2180-2196.
(79) Lesage, S., et al. (2013). Ann Neurol 73, 459-471.
(80) Lettice, L. A., et al. (2003). Hum Mol Genet 12, 1725-1735.
(81) Lewis, J., et al. (2008). Mol Neurodegener 3, 19.
(82) Li, J. Y., et al, (2008). Nat Med 14, 501-503.
(83) Lindvall, O., et al. (1990). Science 247, 574-577.
(84) Lindvall, O., et al. (1989). Archives of neurology 46, 615-631.
(85) Litvan, I., et al. (2007a). J Neuropathol Exp Neurol 66, 329-336.
(86) Litvan, I., et al. (2007b). J Neuropathol Exp Neurol 66, 251-257.
(87) Liu, J., et al. (2006). Hum Mol Genet 15, 1769-1782.
(88) Loots, G. G., et al. (2000). Science 288, 136-140.
(89) Mali, P., et al. (2013). Nature biotechnology 31, 833-838.
(90) Manning-Bog, A. B., et al. (2002). J Biol Chem 277, 1641-1644.
(91) Maraganore, D. M. (2011). J Mov Disord 4, 1-7.
(92) Maraganore, D. M., et al. (2006). Jama 296, 661-670.
(93) Marks, W. J., Jr., et al. (2010). The Lancet Neurology 9, 1164-1172.
(94) Marks, W. J., Jr., et al. (2008). The Lancet Neurology 7, 400-408.
(95) Marlin, S., et al. (1999). Hum Mutat 14, 377-386.
(96) Maston, G. A., et al. (2006). Annu Rev Genomics Hum Genet 7, 29-59.
(97) Matsuda, W., et al. (2009). The Journal of neuroscience: the official journal of the Society for Neuroscience 29, 444-453.
(98) McCormack, A. L., et al. (2010). PLoS One 5, e12122.
(99) Mellick, G. D., et al. (2005). Neurosci Lett 375, 112-116.
(100) Mendez, I., et al. (2008). Nat Med 14, 507-509.
(101) Mizuta, I., et al. (2006). Hum Mol Genet 15, 1151-1158.
(102) Mueller, J. C., et al. (2005). Ann Neurol 57, 535-541.
(103) Nishioka, K., et al. (2006). Ann Neurol 59, 298-309.
(104) Olanow, C. W., et al. (2001). The New England journal of medicine 345, 146; author reply 147.
(105) Olanow, C. W., et al. (2003). Annals of neurology 54, 403-414.
(106) Palfi, S., et al. (2014). Lancet 383, 1138-1146.
(107) Pals, P., et al. (2004). Ann Neurol 56, 591-595.
(108) Perlow, M. J., et al. (1979). Science 204, 643-647.
(109) Pfister, E. L., et al. (2009). Current biology: CB 19, 774-778.
(110) Polymeropoulos, M. H., et al. (1997). Science 276, 2045-2047.
(111) Poulopoulos, M., et al. (2012). Movement disorders: official journal of the Movement
Disorder Society 27, 831-842.
(112) Proukakis, C., et al. (2013). Neurology 80, 1062-1064.
(113) Puschmann, A. (2013). Parkinsonism & related disorders 19, 407-415.
(114) Qi, L. S., et al. (2013). Cell 152, 1173-1183.
(115) Rieder, M. J., et al. (2005). N Engl J Med 352, 2285-2293.
(116) Riederer, P., et al. (1976). Journal of neural transmission 38, 277-301.
(117) Rong, Z., et al. (2014). Protein & cell 5, 258-260.
(118) Ross, O. A., et al. (2007). Mech Ageing Dev 128, 378-382.
(119) Sabherwal, N., et al. (2007). Hum Mol Genet 16, 210-222.
(120) Salegio, E. A., et al. (2012). Advanced drug delivery reviews 64, 598-604.
(121) Sampson, T. R., et al. (2014). Frontiers in cellular and infection microbiology 4, 37.
(122) Scherzer, C. R., et al. (2008). Proc Natl Acad Sci USA 105, 10907-10912.
(123) Schulz-Schaeffer, W. J. (2010). Acta Neuropathol 120, 131-143.
(124) Shelley, B. C., et al. (2014). e51219.
(125) Shen, B., et al. (2014). Nature methods 11, 399-402.
(126) Singleton, A. B., et al. (2003). Science 302, 841.
(127) Smith, C., et al. (2014). Cell stem cell 15, 12-13.
(128) Southgate, T., et al. (2008). Curr Protoc Neurosci Chapter 4, Unit 4 23.
(129) Specht, C. G., et al. (2005). Molecular and cellular neurosciences 28, 326-334.
(130) Sterling, L., et al. (2014). [v1; ref status: awaiting peer review, http://f1000r.es/2pt] F1000Research, 259.
(131) Swiech, L., et al. (2014). Nature biotechnology.
(132) Swistowski, A., et al. (2009). PloS one 4, e6233.
(133) Tan, E. K., et al. (2003). Neurosci Lett 336, 70-72.
(134) Tebas, P., et al. (2014). N Engl J Med 370, 901-910.
(135) Touchman, J. W., et al. (2001). Genome Res 11, 78-86.
(136) Ueda, K., et al. (1993). Proc Natl Acad Sci USA 90, 11282-11286.
(137) Van Den Eeden, S. K., et al. (2003). Am J Epidemiol 157, 1015-1022.
(138) Veres, A., et al. (2014). Cell stem cell 15, 27-30.
(139) Vila, M., et al. (2000). J Neurochem 74, 721-729.
(140) Wang, C. K., et al. (2006). J Neural Transm.
(141) Winkler, S., et al. (2007). Neurology 69, 1745-1750.
(142) Xu, T., et al. (2014). Applied and environmental microbiology 80, 1544-1552.
(143) Zarranz, J. J., et al. (2004). Ann Neurol 55, 164-173.

What is claimed is:

1. A method for treating or preventing a neurodegenerative disease in a human comprising genetically modifying a gene or genomic sequence associated with expression of one or more neuronal proteins associated with a neurodegenerative disease, said method comprising:
(a) administering a genetically engineered vector that includes a gene which encodes a nuclease into a cell in vivo or in vitro; and
(b) expressing a molecular component or a combination of molecular component that will genetically modify a gene in the cell and will alter the expression of a gene in the cell, wherein the gene is associated with a neurodegenerative disease in the human.

2. The method according claim 1, wherein the neurodegenerative disease is an alpha-synucleinopathy comprising Parkinson's disease (PD), Parkinson's disease with dementia (PDD), dementia with Lewy bodies (DLB) or multiple system atrophy (MSA), a tauopathy, for example, frontotemporal dementia (FTD), or Alzheimer's disease, or another form of neurodegenerative disease.

3. The method of claim 1, wherein the gene or genomic sequence is SNCA, MAPT, PSEN1, PARKIN, PINK1, DJ-1, LRRK2, ATP13A2, PLA2G6, FBX07, TAF1, VPS35, EIF4G1, DNAJC6, SYNJ-1, DNAJC13, GCH-1, DCTN1, SLC6A3, GBA, HEXA, SMPD1, POLG, SNCB or a combination thereof.

4. The method of claim 1, wherein the vector permits the expression of a nuclease that introduces a double strand break of genomic DNA.

5. The method of claim 1, wherein the nuclease is selected from a group of nucleases comprising of engineered, non-naturally-occurring zinc-finger nuclease (ZFN), TAL effector nuclease (TALEN), clustered regularly interspaced short palindromic repeat (CRIPSR/Cas9), meganuclease, and a combination thereof.

6. The method of claim 1, wherein the nuclease recognizes one or more gene specific non-naturally occurring guide polynucleotide (e.g. RNA) that hybridises with a gene at one or more specific site.

7. The method of claim 6, wherein a plurality of guide polynucleotides are incorporated with a plurality of nucleases in a multiplexing step.

8. The method of claim 1, wherein the nuclease recognizes one or more gene specific non-naturally occurring guide polypeptide (e.g. protein) that hybridises with a gene at one or more specific site.

9. The method of claim 8, wherein a plurality of guide polypeptides are incorporated with a plurality of nucleases in a multiplexing step.

10. The method of claim 1, wherein the genetically engineered vector comprises one or more vectors and components and is located on the same vector or different vectors of the system, whereby the guide polynucleotide or guide polypeptide targets a gene or a genomic sequence.

11. The method of claim 1, wherein administering the vector comprises delivering the vector via a viral host to the cell, and wherein the cell comprises of a neuron or a brain-related cell.

12. The method of claim 11, wherein the viral host is selected from the list comprising of adeno-associated viral (AAV) based system, a lentiviral based system, a retroviral based system, and/or a combination thereof.

13. The method of claim 1, wherein administering the vector comprises delivering the vector via a viral host to the cell, and wherein the cell is a stem cell.

14. The method of claim 13, wherein the stem cell is selected from the group comprising of embryonic stem cell (hESC), HLA-typed induced pluripotent stem cell (iPSC), pluripotent stem cell clone derived from a patient, or a combination thereof.

15. The method of claim 13, wherein the stem cell after being administered with the vector differentiates into a neuron or neuronal progenitor cell or a brain-related cell and further being transplanted into a brain region of a human.

16. The method of claim 1, wherein the nuclease introduces a double-strand break guided by a molecular component.

17. The method of claim 16, wherein the molecular component is a polynucleotide or a polypeptide.

18. The method of claim 16, wherein the molecular component mediates non-homologous end joining.

19. The method of claim 16, wherein the molecular component mediates homology directed repair.

20. The method of claim 16, wherein the molecular component targets regulatory element of the gene and induces a genomic deletion.

21. The method of claim 16, wherein the molecular component targets splice sites of the gene and induces a genomic deletion.

22. The method of claim 16, wherein the molecular component targets the promoter region of the gene and inhibits expression of the gene.

23. The method of claim 16, wherein the molecular component targets the regulatory element of the gene and inhibits expression of the gene.

24. The method of claim 16, wherein the molecular component targets the promoter region of the gene and increases expression of the gene.

25. The method of claim 16, wherein the molecular component targets the regulatory element of the gene and increases expression of the gene.

26. A medical composition for treating or preventing a neurodegenerative disease in a human comprising:
(a) a viral delivery system that delivers the nuclease and molecular component to a cell; and
(b) a nuclease guided by a molecular component to target a gene associated with a neurodegenerative disease.

27. The medical composition of claim 26, wherein the neurodegenerative disease comprises an alpha-synucleinopathy, such as Parkinson's disease (PD), Parkinson's disease with dementia (PDD), dementia with Lewy bodies (DLB) or multiple system atrophy (MSA), a tauopathy, for example, frontotemporal dementia (FTD), or Alzheimer's disease, or another form of neurodegenerative disease associated therewith.

28. The medical composition of claim 26, wherein the gene or genomic sequence comprises one or more of the following sequences: SNCA, MAPT, PSEN1, PARKIN, PINK1, DJ-1, LRRK2, ATP13A2, PLA2G6, FBX07, TAF1, VPS35, EIF4G1, DNAJC6, SYNJ-1, DNAJC13, GCH-1, DCTN1, SLC6A3, GBA, HEXA, SMPD1, POLG, SNCB or a combination thereof.

29. The medical composition of claim 26, wherein the delivery system is mediated by a viral host.

30. The medical composition of claim 29, wherein the viral host is an adeno-associated viral (AAV) based system, a lentiviral based system, a retroviral based system, or a combination thereof.

31. The medical composition of claim 26, wherein the nuclease comprises a zinc-finger nuclease (ZFN), TAL effector nuclease (TALEN), clustered regularly interspaced short palindromic repeat (CRIPSR/Cas9), meganuclease, or a combination thereof.

32. The medical composition of claim 26, wherein the molecular component is a non-naturally occurring polynucleotide or a polypeptide that hybridises with a gene or genomic sequence.

33. The medical composition of claim 26, wherein the nuclease recognizes one or more gene specific guide polynucleotides that hybridise with a gene as listed in claim 28.

34. The medical composition of claim 26, wherein the nuclease recognizes one or more gene specific guide polypeptides that hybridise with a gene as listed in claim 28.

35. The medical composition of claim 26, wherein the nuclease recognizes one or more gene specific guide polynucleotides that hybridise with at least one or more regions of an alpha-synuclein (SNCA) gene, wherein the region of the SNCA gene is exon 2, exon 3, exon 4, exon 5, a promoter, a splice-site, or a regulatory domain of SNCA, a non-coding genomic region of SNCA, or a combination thereof.

36. The medical composition of claim 29, wherein the nuclease recognizes one or more gene specific guide polynucleotides that hybridise with a microtubule-associated protein tau (MAPT) gene.

37. The medical composition of claim 26, wherein the nuclease recognizes one or more gene specific guide polynucleotide that hybridises with a region of a microtubule associated protein tau (MAPT) gene, wherein the region is an exon of MAPT, a regulatory domain of MAPT, a non-coding genomic region of MAPT, or a combination thereof.

38. The medical composition of claim 26, wherein the nuclease introduces a mutation in the gene, and wherein the mutation leads to expression of a non-functional protein.

39. The medical composition of claim 26, wherein the nuclease gene is Cas9.

40. The medical composition of claim 26, wherein Cas 9 is a mutant Cas 9 gene that encodes a catalytically inactive protein fused to a VP16 tetramer activation domain or a Kruppel-associated box repressor domain.

41. The medical composition of claim 26, wherein the guide polynucleotide guides the nuclease to a targeted gene mediated by hybridization to provide a double strand break or a modulation of gene expression.

42. A fusion protein comprising a CRISPR/Cas 9 nuclease component and a guide polynucleotide targeting the SNCA gene comprising an exonic region, wherein said exonic region comprises a sequence of (SEQ ID NO: 1-SEQ ID NO: 9), a regulatory element wherein said regulatory element comprises a sequence (SEQ ID NO: 13-SEQ ID NO: 22), a promoter region comprising a sequence (SEQ ID NO: 23-SEQ ID NO: 62), an intronic region comprising a sequence (SEQ ID NO: 63-SEQ ID NO: 87), or combinations thereof.

43. The fusion protein of claim 42, wherein the polynucleotide encodes an isolated fusion protein.

44. The fusion protein of claim 42, wherein the fusion protein is combined with an isolated cell which in itself comprises the polynucleotide(s).

45. The fusion protein of claim 44, wherein the isolated cell is a stem cell.

46. The fusion protein of claim 44, wherein the isolated cell is a brain-related cell.

47. The fusion protein of claim 42 further comprising a pharmaceutically acceptable excipient.

48. The fusion protein of claim 42, comprising the polynucleotide and a pharmaceutically acceptable excipient.

49. The medical composition of claim 26, wherein the SNCA gene is modified by a method comprising introducing one or more fusion proteins of claim 42 into a cell.

50. A fusion protein comprising a CRISPR/Cas 9 nuclease component and a guide polynucleotide targeting MAPT gene inclusive of exonic, intronic and regulatory elements comprise SEQ ID NO:10, SEQ ID NO:11, SEQ ID NO: 12, or a combination thereof.

51. The medical composition of claim 49, wherein the polynucleotide encodes an isolated fusion protein.

52. The medical composition of claim 49, wherein the fusion protein is combined with an isolated cell which in itself comprises the polynucleotide(s).

53. The medical composition of claim 49, wherein the isolated cell is a stem cell.

54. The medical composition of claim 49, wherein the isolated cell is a brain-related cell.

55. The medical composition of claim 49, comprising the fusion protein and a pharmaceutically acceptable excipient.

56. The medical composition of claim 49, comprising the polynucleotide and a pharmaceutically acceptable excipient.

57. The medical composition of claim 49, wherein the MAPT gene is modified by a method comprising introducing one or more fusion proteins of claim 51 into a cell.


SEQUENCE LISTING (87 sequences)

SEQ ID NO: 1 - DNA, 23 bp, Homo sapiens
gtaaaggaattcattagccatgg

SEQ ID NO: 2 - DNA, 23 bp, Homo sapiens
gccatggatgtattcatgaaagg

SEQ ID NO: 3 - DNA, 23 bp, Homo sapiens
tcctttcatgaatacatccatgg

SEQ ID NO: 4 - DNA, 23 bp, Homo sapiens
accaaggagggagtggtgcatgg

SEQ ID NO: 5 - DNA, 23 bp, Homo sapiens
accatgcaccactccctccttgg

SEQ ID NO: 6 - DNA, 23 bp, Homo sapiens
ctttgtcaaaaaggaccagttgg

SEQ ID NO: 7 - DNA, 23 bp, Homo sapiens
gagcaagtgacaaatgttggagg

SEQ ID NO: 8 - DNA, 23 bp, Homo sapiens
gcctcattgtcaggatccacagg

SEQ ID NO: 9 - DNA, 23 bp, Homo sapiens
gcctgtggatcctgacaatgagg

SEQ ID NO: 10 - DNA, 23 bp, Homo sapiens
cacgctgggacgtacgggttggg

SEQ ID NO: 11 - DNA, 23 bp, Homo sapiens
aagatcacgctgggacgtacggg

SEQ ID NO: 12 - DNA, 23 bp, Homo sapiens
atcacttcgaactcctggcgggg

SEQ ID NO: 13 - DNA, 23 bp, Homo sapiens
tgtgaaagaccgtaagttgctgg

SEQ ID NO: 14 - DNA, 23 bp, Homo sapiens
ggatcaagacaacttggccatgg

SEQ ID NO: 15 - DNA, 23 bp, Homo sapiens
aagaggtgagttctaatcattgg

SEQ ID NO: 16 - DNA, 23 bp, Homo sapiens
cttgatggcagtcacttgaaagg

SEQ ID NO: 17 - DNA, 23 bp, Homo sapiens
atggtgtcctgagatgactgagg

SEQ ID NO: 18 - DNA, 23 bp, Homo sapiens
gtcaagtttactatcttacgtgg

SEQ ID NO: 19 - DNA, 23 bp, Homo sapiens
tttcacttacgtactatgaaagg

SEQ ID NO: 20 - DNA, 23 bp, Homo sapiens
gaattgtactgactacaccacgg

SEQ ID NO: 21 - DNA, 23 bp, Homo sapiens
tactcatattacagtcagtctgg

SEQ ID NO: 22 - DNA, 23 bp, Homo sapiens
atccttgtataaaccccacaagg

SEQ ID NO: 23 - DNA, 23 bp, Artificial Sequence
  Description of Artificial Sequence Synthetic oligonucleotide
tagccacctaaccacggattagg

SEQ ID NO: 24 - DNA, 23 bp, Artificial Sequence
  Description of Artificial Sequence Synthetic oligonucleotide
ggttaacaagtgctggcgcgggg

SEQ ID NO: 25 - DNA, 23 bp, Artificial Sequence
  Description of Artificial Sequence Synthetic oligonucleotide
gggttaacaagtgctggcgcggg

SEQ ID NO: 26 - DNA, 23 bp, Artificial Sequence
  Description of Artificial Sequence Synthetic oligonucleotide
catcctaatccgtggttaggtgg

SEQ ID NO: 27 - DNA, 23 bp, Artificial Sequence
  Description of Artificial Sequence Synthetic oligonucleotide
cgggttaacaagtgctggcgcgg

SEQ ID NO: 28 - DNA, 23 bp, Artificial Sequence
  Description of Artificial Sequence Synthetic oligonucleotide
agttggatgctcacgctccatgg

SEQ ID NO: 29 - DNA, 23 bp, Artificial Sequence
  Description of Artificial Sequence Synthetic oligonucleotide
tggagcatcctcgcgtttcccgg

SEQ ID NO: 30 - DNA, 23 bp, Artificial Sequence
  Description of Artificial Sequence Synthetic oligonucleotide
gagcatcctcgcgtttcccgggg

SEQ ID NO: 31 - DNA, 23 bp, Artificial Sequence
  Description of Artificial Sequence Synthetic oligonucleotide
gcttttccccgggaaacgcgagg

SEQ ID NO: 32 - DNA, 23 bp, Artificial Sequence
  Description of Artificial Sequence Synthetic oligonucleotide
attcatcctaatccgtggttagg

SEQ ID NO: 33 - DNA, 23 bp, Artificial Sequence
  Description of Artificial Sequence Synthetic oligonucleotide
agaacgccccctcgggtggctgg

SEQ ID NO: 34 - DNA, 23 bp, Artificial Sequence
  Description of Artificial Sequence Synthetic oligonucleotide
tgctggcgcggggtccgctaggg

SEQ ID NO: 35 - DNA, 23 bp, Artificial Sequence
  Description of Artificial Sequence Synthetic oligonucleotide
tggcgcggggtccgctagggtgg

SEQ ID NO: 36 - DNA, 23 bp, Artificial Sequence
  Description of Artificial Sequence Synthetic oligonucleotide
agaggattcatcctaatccgtgg

SEQ ID NO: 37 - DNA, 23 bp, Artificial Sequence
  Description of Artificial Sequence Synthetic oligonucleotide
ggtaacgggttaacaagtgctgg

SEQ ID NO: 38 - DNA, 23 bp, Artificial Sequence
  Description of Artificial Sequence Synthetic oligonucleotide
gcaggcgccgctcacactcgcgg

SEQ ID NO: 39 - DNA, 23 bp, Artificial Sequence
  Description of Artificial Sequence Synthetic oligonucleotide
caggcgccgctcacactcgcggg

SEQ ID NO: 40 - DNA, 23 bp, Artificial Sequence
  Description of Artificial Sequence Synthetic oligonucleotide
tcccggggaaaagcggatcccgg

SEQ ID NO: 41 - DNA, 23 bp, Artificial Sequence
  Description of Artificial Sequence Synthetic oligonucleotide
ggagcatcctcgcgtttcccggg

SEQ ID NO: 42 - DNA, 23 bp, Artificial Sequence
  Description of Artificial Sequence Synthetic oligonucleotide
actgttaccctttagaccccggg

SEQ ID NO: 43 - DNA, 23 bp, Artificial Sequence
  Description of Artificial Sequence Synthetic oligonucleotide
tttaaattcccggggtctaaagg

SEQ ID NO: 44 - DNA, 23 bp, Artificial Sequence
  Description of Artificial Sequence Synthetic oligonucleotide
gctgagaacgccccctcgggtgg

SEQ ID NO: 45 - DNA, 23 bp, Artificial Sequence
  Description of Artificial Sequence Synthetic oligonucleotide
ctaactgctcactcggggtgtgg

SEQ ID NO: 46 - DNA, 23 bp, Artificial Sequence
  Description of Artificial Sequence Synthetic oligonucleotide
agacggcccgcgagtgtgagcgg

SEQ ID NO: 47 - DNA, 23 bp, Artificial Sequence
  Description of Artificial Sequence Synthetic oligonucleotide
ttaaattcccggggtctaaaggg

SEQ ID NO: 48 - DNA, 23 bp, Artificial Sequence
  Description of Artificial Sequence Synthetic oligonucleotide
cagaactaactgctcactcgggg

SEQ ID NO: 49 - DNA, 23 bp, Artificial Sequence
  Description of Artificial Sequence Synthetic oligonucleotide
tcggggtgtggttcaaactcagg

SEQ ID NO: 50 - DNA, 23 bp, Artificial Sequence
  Description of Artificial Sequence Synthetic oligonucleotide
taaagtaagcaagctgcgtttgg

SEQ ID NO: 51 - DNA, 23 bp, Artificial Sequence
  Description of Artificial Sequence Synthetic oligonucleotide
ggaaacgcgaggatgctccatgg

SEQ ID NO: 52 - DNA, 23 bp, Artificial Sequence
  Description of Artificial Sequence Synthetic oligonucleotide
tcgcgtttcccggggaaaagcgg

SEQ ID NO: 53 - DNA, 23 bp, Artificial Sequence
  Description of Artificial Sequence Synthetic oligonucleotide
tactgttaccctttagaccccgg

SEQ ID NO: 54 - DNA, 23 bp, Artificial Sequence
  Description of Artificial Sequence Synthetic oligonucleotide
accccgcgccagccacccgaggg

SEQ ID NO: 55 - DNA, 23 bp, Artificial Sequence
  Description of Artificial Sequence Synthetic oligonucleotide
aaagtctagccacctaaccacgg

SEQ ID NO: 56 - DNA, 23 bp, Artificial Sequence
  Description of Artificial Sequence Synthetic oligonucleotide
gtgtacactcatttaaccattgg

SEQ ID NO: 57 - DNA, 23 bp, Artificial Sequence
  Description of Artificial Sequence Synthetic oligonucleotide
tgaattcctttacaccacactgg

SEQ ID NO: 58 - DNA, 23 bp, Artificial Sequence
  Description of Artificial Sequence Synthetic oligonucleotide
ggaggctgagaacgccccctcgg

SEQ ID NO: 59 - DNA, 23 bp, Artificial Sequence
  Description of Artificial Sequence Synthetic oligonucleotide
gaggctgagaacgccccctcggg

SEQ ID NO: 60 - DNA, 23 bp, Artificial Sequence
  Description of Artificial Sequence Synthetic oligonucleotide
cccctcgggtggctggcgcgggg

SEQ ID NO: 61 - DNA, 23 bp, Artificial Sequence
  Description of Artificial Sequence Synthetic oligonucleotide
ggattagaaccatcacacttggg

SEQ ID NO: 62 - DNA, 23 bp, Artificial Sequence
  Description of Artificial Sequence Synthetic oligonucleotide
aatctgtctgcccgctctcttgg

SEQ ID NO: 63 - DNA, 23 bp, Artificial Sequence
  Description of Artificial Sequence Synthetic oligonucleotide
ttgccgcaatgtttccccggagg

SEQ ID NO: 64 - DNA, 23 bp, Artificial Sequence
  Description of Artificial Sequence Synthetic oligonucleotide
gcacaccttaatattactactgg

SEQ ID NO: 65 - DNA, 23 bp, Artificial Sequence
  Description of Artificial Sequence Synthetic oligonucleotide
gggaaacattgcggcaacatggg

SEQ ID NO: 66 - DNA, 23 bp, Artificial Sequence
  Description of Artificial Sequence Synthetic oligonucleotide
ggggaaacattgcggcaacatgg

SEQ ID NO: 67 - DNA, 23 bp, Artificial Sequence
  Description of Artificial Sequence Synthetic oligonucleotide
tgtgccattttcaagatccgtgg

SEQ ID NO: 68 - DNA, 23 bp, Artificial Sequence
  Description of Artificial Sequence Synthetic oligonucleotide
taaactccacaatgcctccgggg

SEQ ID NO: 69 - DNA, 23 bp, Artificial Sequence
  Description of Artificial Sequence Synthetic oligonucleotide
atgttgccgcaatgtttccccgg

SEQ ID NO: 70 - DNA, 23 bp, Artificial Sequence
  Description of Artificial Sequence Synthetic oligonucleotide
atgcctccggggaaacattgcgg

SEQ ID NO: 71 - DNA, 23 bp, Artificial Sequence
  Description of Artificial Sequence Synthetic oligonucleotide
ttggccacggatcttgaaaatgg

SEQ ID NO: 72 - DNA, 23 bp, Artificial Sequence
  Description of Artificial Sequence Synthetic oligonucleotide
aatcttacatatagggatgttgg

SEQ ID NO: 73 - DNA, 23 bp, Artificial Sequence
  Description of Artificial Sequence Synthetic oligonucleotide
tctaaactccacaatgcctccgg

SEQ ID NO: 74 - DNA, 23 bp, Artificial Sequence
  Description of Artificial Sequence Synthetic oligonucleotide
atgtttccccggaggcattgtgg

SEQ ID NO: 75 - DNA, 23 bp, Artificial Sequence
  Description of Artificial Sequence Synthetic oligonucleotide
ctaaactccacaatgcctccggg

SEQ ID NO: 76 - DNA, 23 bp, Artificial Sequence
  Description of Artificial Sequence Synthetic oligonucleotide
gaatgccagtagtaatattaagg

SEQ ID NO: 77 - DNA, 23 bp, Artificial Sequence
  Description of Artificial Sequence Synthetic oligonucleotide
acatatagggatgttggccacgg

SEQ ID NO: 78 - DNA, 23 bp, Artificial Sequence
  Description of Artificial Sequence Synthetic oligonucleotide
ctctctacatgctcattacgtgg

SEQ ID NO: 79 - DNA, 23 bp, Artificial Sequence
  Description of Artificial Sequence Synthetic oligonucleotide
cttactacttgaccctttacagg

SEQ ID NO: 80 - DNA, 23 bp, Artificial Sequence
  Description of Artificial Sequence Synthetic oligonucleotide
agtaagtatattaagctatgtgg

SEQ ID NO: 81 - DNA, 23 bp, Artificial Sequence
  Description of Artificial Sequence Synthetic oligonucleotide
gtcatcccattggacatgtatgg

SEQ ID NO: 82 - DNA, 23 bp, Artificial Sequence
  Description of Artificial Sequence Synthetic oligonucleotide
tttactatgcgtcatcccattgg

SEQ ID NO: 83 - DNA, 23 bp, Artificial Sequence
  Description of Artificial Sequence Synthetic oligonucleotide
ttttgccatacatgtccaatggg

SEQ ID NO: 84 - DNA, 23 bp, Artificial Sequence
  Description of Artificial Sequence Synthetic oligonucleotide
tttttgccatacatgtccaatgg

SEQ ID NO: 85 - DNA, 23 bp, Artificial Sequence
  Description of Artificial Sequence Synthetic oligonucleotide
ggaaatcaatggaatgccaaagg

SEQ ID NO: 86 - DNA, 23 bp, Artificial Sequence
  Description of Artificial Sequence Synthetic oligonucleotide
tggaaagttagggaaatcaatgg

SEQ ID NO: 87 - DNA, 23 bp, Artificial Sequence
  Description of Artificial Sequence Synthetic oligonucleotide
atcaatggaatgccaaaggaagg